(19) United States
(12) Patent Application Publication
(10) Pub. No.: US 2008/0181847 A1
(43) Pub. Date: Jul. 31, 2008

(54) Targeted Imaging and/or Therapy Using the Staudinger Ligation

(75) Inventors: Marc Stefan Robillard, Eindhoven (NL); Holger Gruell, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/576,536

(22) PCT Filed: Oct. 4, 2005
(86) PCT No.: PCT/IB05/53258

§ 371 Date: Apr. 3, 2007

(30) Foreign Application Priority Data
Oct. 7, 2004 (EP) 04104913.1

(51) Int. Cl. A61K 51/00; A61K 39/395; A61K 49/06; C40B 50/00; C40B 30/04; C40B 40/10; C07D 257/02; A61K 49/04

(52) U.S. Cl. 424/1.11; 424/130.1; 424/9.3; 424/9.4; 424/1.77; 540/474; 506/23; 506/18; 506/9

(57) ABSTRACT

The use of a selective chemical and bioorthogonal reaction providing a covalent ligation such as the Staudinger ligation, in targeted molecular imaging and therapy is presented, more specifically with interesting applications for pre-targeted imaging or therapy. Current pre-targeted imaging is hampered by the fact that it relies solely on natural/biological targeting constructs (i.e. biotin/streptavidin). Size considerations and limitations associated with their endogenous nature severely limit the number of applications. The present invention describes how the use of an abiotic, bio-orthogonal reaction which forms a stable adduct under physiological conditions, by way of a small or undetectable bond, can overcome these limitations.

The present invention relates to novel compounds, kits and methods, for use in medical imaging and therapy. The present invention also relates to novel compounds and kits for pre-targeted imaging and/or therapy and to methods of production and use thereof

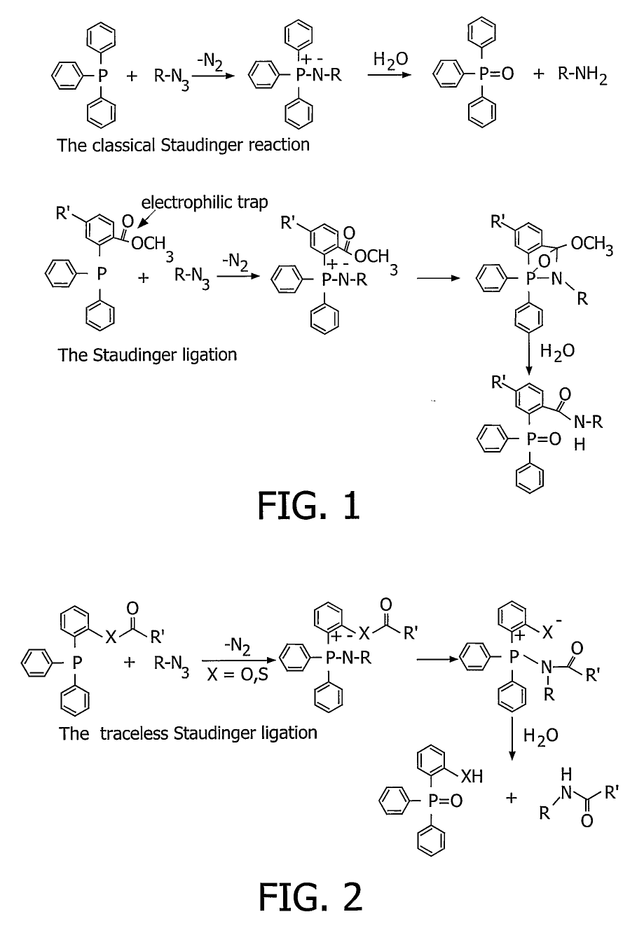

A chemoselective ligation, based on the classical Staudinger reaction between an azide and a phosphine (scheme 1 of FIG. 1), was applied by Bertozzi and co-workers to study cell surface glycosylation [reviewed in Kohn & Breinbauer (2004) Angew. Chem. Int. Ed. 43, 3106-3116].

A further modification is called the traceless Staudinger ligation and is depicted in FIG. 2. Using the Staudinger ligation, Bertozzi and co-workers have demonstrated that N-azidoacetylmannosamine (ManNAz) was metabolically converted to the corresponding sialic acid and incorporated into cell surface glycoconjugates. The azide was available on the cell surface for Staudinger ligation with exogenous phosphine reagents. Control experiments revealed that neither azide reduction by endogenous monothiols (such as glutathione) nor the reduction of disulfides on the cell surface by the phosphine probe takes place.

Applications of this technique (“metabolic interference”) include the engineering of the composition of cell surfaces by chemically constructing new glycosylation patterns on cells (probing glycosylation function). The reaction has also been used for tagging within a cellular environment. For instance, azides are incorporated into proteins via unnatural amino acids and these proteins are targeted for covalent modification within cellular lysates [Kiick et al. (2002) Proc. Natl. Acad. Sci. 99, 19-24]. Azidohomoalanine was activated by the methionyl-t-RNA synthetase of E coli and replaced methionine when the protein was expressed in methionine-depleted bacterial cultures. One application is the protein modification with a pro-fluorescent coumarin dye activated by the Staudinger ligation, allowing the imaging of protein trafficking within cells [Lemieux et al. (2003) J. Am. Chem. Soc. 125, 4708-4709].

The Staudinger ligation has successfully been used for numerous goals, such as peptide ligation, lactam synthesis, bioconjugates, intracellular tagging, metabolic cell engineering, and the production of micro-arrays. The Staudinger Ligation has been shown to proceed as well in vivo (rats) and the azide and phosphine derivatives proved non-toxic in vitro and in vivo [Prescher et al. (2004) Nature 430, 873-877]. The general usefulness of this reaction for molecular imaging has remained largely unexplored.

In medical imaging modalities, the use of contrast agents (materials which enhance image contrast, for example between different organs or tissues or between normal and abnormal tissue) is well established. The imaging of specific molecular targets that are associated with disease allows earlier diagnosis and better management of disease. Of particular interest, therefore, are contrast agents that distribute preferentially to distinct body sites, e.g. tumor cells, by virtue of active targeting. Such active targeting is achieved by the direct or indirect conjugation of a contrast-enhancing moiety to a targeting construct. The targeting construct binds to cell surfaces or other surfaces at the target site or is taken up by the cell.

An important criterion for a successful imaging agent for use on living humans and animals is that it exhibits a high target uptake while showing a rapid clearance (through renal and/or hepatobiliary systems) from non-target tissue and from the blood, so that a high contrast between the target and surrounding tissues can be obtained. However, this is often problematic. For example, imaging studies in humans have shown that the maximum concentration of antibody at the tumor site is attainable within 24 h but that several more days are required before the concentration of a labeled antibody in circulation decreases to levels low enough for successful imaging to take place. This is in particular a challenge for nuclear probes, because these constantly produce signal by decaying. Consequently, a sufficient signal to background level has to be reached within several half-lives of the tracer. For MRI probes one could wait long enough for the background signal to diminish before imaging. Also activatable probes or “smart probes” exist for MRI approaches; these produce signal only when they interact with a target or enzyme (see U.S. Pat. No. 6,770,261). However, endogenous receptor densities are often too low for sufficient signal accumulation for MRI.

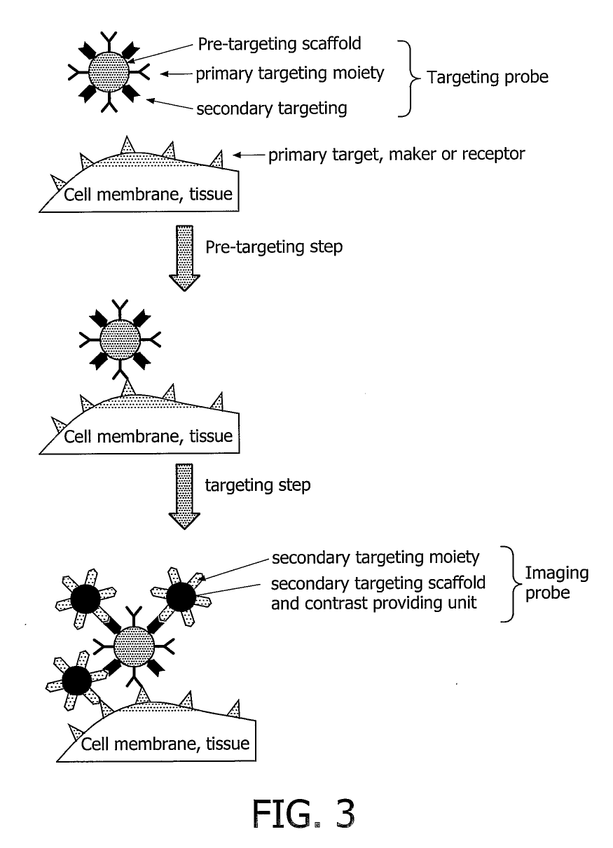

These problems with slow or insufficient accumulation in target tissue, slow clearance from non-target areas and low contrast agent concentration (especially for MRI) have lead to the application of pre-targeting schemes. FIG. 3 shows a typical pre-targeting scheme. In the pre-targeting step, a primary target, such as a receptor of interest, is selectively identified by way of a primary targeting moiety. In order to allow detection after binding, the primary targeting moiety is linked to a pre-targeting scaffold which also carries a secondary target. In a second targeting step, a secondary targeting moiety is administered which will bind to the secondary target on the pre-targeting scaffold. This secondary targeting moiety is itself bound to a secondary targeting scaffold which holds a contrast providing unit. Typical examples of secondary target/secondary targeting moiety pairs are biotin/streptavidin or antigen/antibody systems.

There are several problems and disadvantages associated with current (pre)targeted imaging. The main issue being that targeting relies solely on natural/biological targeting constructs (i.e. endogenous receptors, biotin/streptavidin). This leads to a range of drawbacks in particular with respect to size and their endogenous nature.

The entities that carry out highly selective interactions in biology in general (like antibody-antigen), and in pre-targeting in particular (biotin-streptavidin, oligonucleotides as secondary targeting moieties), are very large. Due to the size, the pre-targeting concept is so far basically limited to applications within the vascular system. As a result, pre-targeting with peptides and small organic targeting devices as primary ligands, as well as with metabolic imaging and intracellular target imaging, have remained out of reach as the size of the secondary targeting moieties precludes the use of small primary ligands. The bulky secondary targeting moieties affect the properties (i.e. transport, elimination, target affinity/interaction) of the pre-targeting construct as well as the imaging probe. Also, the contrast-providing unit of the imaging probe can affect the properties of the secondary targeting moieties (e.g. loss of affinity of biotin conjugate for avidin).

Furthermore, a number of compounds which are used for pre-targeting are degraded by the body. Biotin is an endogenous molecule and its conjugates can be cleaved by the serum enzyme biotinidase. When antisense pre-targeting is used, the oligonucleotides are subject to attack by RNAse and DNAse. Proteins and peptides are also subject to natural decomposition pathways.

The interactions between the respective partners can be further impaired by their non-covalent and dynamic nature. Also, endogenous biotin competes with biotin conjugates for streptavidin binding. Streptavidin can induce immune reactions. And finally, naturally occurring targets like cell surface receptors are not always present in sufficient amounts to create contrast during imaging.

The technique of pre-targeting has proven very useful for antibody-based imaging, since their pharmacokinetics are usually too slow for imaging applications despite the high selectivity and specificity for their antigens [Sung et al. (1992), Cancer Res. 52, 377-384; Juweid et al. (1992) Cancer Res. 52, 5144-5153]. Although smaller targeting constructs such as antibody fragments, peptides and organic molecules have more appropriate pharmacokinetics, they could profit from a pre-targeting approach as well, since these constructs still suffer from slow targeting and clearance (i.e. in dense tissues, or with intracellular imaging) or insufficient accumulation (low receptor density, slow growing or small tumors). Furthermore, accumulation in the clearance pathway, like hepatobiliary or kidney, can obscure the tissue of interest.

A recent development in the imaging field is the move towards generalized tracers in which the labeling chemistry remains largely unchanged, but the underlying molecular structure can be easily modified to image a new molecular target. This would afford a reduction in development time/cost for a new imaging agent. Pre-targeting approaches could allow such a generalization to many targets as the contrast-providing group stays always the same for different applications. Consequently, a faster FDA approval of a new molecular imaging application can be expected, as only the pre-targeting group needs FDA approval.

The present invention provides probes and precursors, kits of probes and precursors, methods of producing such probes and precursors, and methods of applying probes and precursors in the context of medical imaging and therapy.

In its broadest aspect, the present invention relates to two components which interact with each other to form a stable covalent bio-orthogonal bond. These components are of use in medical imaging and therapy, more particularly in targeted and pre-targeted imaging and therapy.

According to a particular embodiment of the invention the covalent bio-orthogonal bond is obtained by the Staudinger ligation, and each of the components of the invention comprise a reaction partner for the Staudinger ligation, i.e. a phosphine and an azide group, respectively.

A first aspect of the invention relates to the two components, e.g. as present in a kit. The kit of the invention comprises at least one targeting probe, comprising a primary targeting moiety and a secondary target and at least one further probe which is an imaging probe, comprising a secondary targeting moiety and a label. Alternatively, the second component is a therapeutic probe, comprising a secondary targeting moiety and a pharmaceutically active compound. According to the invention one of the targeting probe or the imaging or therapeutic probe comprises, as secondary target and secondary targeting moiety respectively, either at least one azide group and the other probe comprises at least one phosphine group, said phosphine and said azide groups being reaction partners for the Staudinger ligation.

Particular embodiments of the invention relate to targeting probes wherein the primary targeting moieties bind to a component either within or outside the vascular system, or specifically either to a component in the interstitial space or to an intracellular component.

Particular embodiments of suitable primary targeting moieties for use in the kits of the present invention are described herein and include receptor binding peptides and antibodies. A particular embodiment of the present invention relates to the use of small targeting moieties, such as peptides, so as to obtain a cell-permeable targeting probe.

A further aspect of the invention relates to a method for developing targeting probes for use in the context of the present invention. A particular embodiment of this aspect of the invention relates to the production of a targeting probe for targeting a receptor by way of combinatorial chemistry, whereby the azide group is introduced during the synthesis of the compound library. More particularly, the present invention relates to a method of developing a targeting probe with optimal binding affinity for a target and optimal reaction with an imaging or therapeutic probe, which comprises making a compound library of the targeting moiety of said targeting probe, whereby the secondary target is introduced at different sites on said targeting moiety, and screening the so obtained compound library for binding with the target and with an imaging and/or therapeutic probe. Thus the present invention also provides libraries of lead targeting moieties modified with at least one azide group at the same or different amino acids. The invention further provides a library of derivatives or variations of a specific peptide characterized in that the derivatives are modified with an azide group at different amino acid positions in the amino acid chain of said peptide.

Further particular embodiments of the invention relate to a kit of the above-described targeting probes and one or more imaging probes and/or therapeutic probes and the use thereof. Such an imaging probe will comprise, in addition to the secondary targeting moiety which is a reaction partner in the bio-orthogonal reaction of the present invention, a detectable label, particularly a contrast agent used in traditional imaging systems, selected from the group consisting of MRI-imageable agents, spin labels, optical labels, e.g. luminescent, bioluminescent and chemoluminescent labels, FRET-type labels and Raman-type labes, ultrasound-responsive agents, X-ray-responsive agents, radionuclides for SPECT (single photon emission computed tomography) and PET (Positron Emission Tomography), suitable examples of which are known to the skilled person and are provided herein.

A particular embodiment of the present invention relates to the use of small size organic PET and SPECT labels as detectable labels, which provide for cell-permeable imaging probes.

Another particular embodiment of the present invention relates to imaging probes which comprise a “smart” or “responsive” contrast agent for MRI as detectable label and their use in the kits and methods of the present invention. More particularly, the present invention relates to an imaging probe comprising an imaging agent for MRI and a phosphine group, which can react with an azide in a Staudinger ligation, whereby a metal atom of the imaging agent is coordinated with carboxylic acid or acids via a link containing the phosphine group.

Further particular embodiments of the invention relate to a kit of the above-described targeting probes and a therapeutic probe, and its use in targeted therapy.

The imaging probe of the present invention can optionally also comprise a therapeutically active compound, which can for instance be a drug or a radioactive isotope. Alternatively, the imaging probe can, in addition to the detectable label comprise a therapeutically active compound.

Another aspect of the invention provides probes particularly suited for medical imaging. Thus the invention provides an imaging probe comprising a secondary targeting moiety and a label whereby the imaging probe comprises as secondary targeting moiety at least one azide group or at least one phosphine group, the phosphine or azide groups being suitable reaction partners for the Staudinger ligation and the label being an imaging label suitable for imaging using classical techniques including MRI, X-ray, ultrasound and the like.

A further aspect of the present invention relates to a combined probe for medical imaging and/or therapy comprising a primary targeting moiety and a detectable label or pharmaceutically active compound whereby the targeting moiety is connected to the detectable label via an amide bond or a triphenylphosphine oxide moiety. In a particular embodiment of this aspect of the invention, the primary targeting moiety is a peptide.

The present invention further relates to a method of in vitro preparing a combined targeting and imaging or therapeutic probe, comprising a primary targeting moiety and a detectable label or a pharmaceutically active agent, comprising the step of reacting a phosphine-comprising detectable label with an azide-comprising primary targeting moiety or reacting an azide-comprising detectable label with a phosphine-comprising primary targeting moiety.

The invention further relates to a method of developing a combined probe for medical imaging or therapy with optimal binding affinity for a target and optimal imaging or therapeutic efficiency, which comprises making a compound library of the targeting moiety of the combined probe, whereby the secondary target is introduced at different sites on said targeting moiety, linking the compounds of said library to a label or pharmaceutically active compound and screening the so obtained compound library for binding with the target and/or for therapeutic efficiency. This method is particularly suited for combined probes wherein the primary targeting moiety is a peptide or a protein.

In a further aspect of the invention the two components which interact in the bio-orthogonal covalent reaction are a target metabolic precursor on the one hand and an imaging or therapeutic probe on the other hand.

Thus in a particular embodiment the present invention provides kits for targeted medical imaging or therapeutics comprising at least one target metabolic precursor comprising a secondary target and at least one further probe selected from either an imaging probe comprising a secondary targeting moiety and a label or a therapeutic probe comprising a secondary targeting moiety and a pharmaceutically active compound, whereby one of the target metabolic substrate or the imaging or therapeutic probe comprises, as secondary target and secondary targeting moiety respectively, either at least one azide group and the imaging or therapeutic probe comprises at least one phosphine group, the phosphine and the azide groups being reaction partners for the Staudinger ligation.

Particular embodiments of this aspect of the invention relate to the above described kits wherein the target metabolic precursor comprises the at least one azide group and wherein the imaging or therapeutic probe comprises the at least one phosphine group.

Further particular embodiments of the present invention relate to kits comprising a metabolic precursor which is selected from a group consisting of sugars, amino acids, nucleobases and choline.

Further particular embodiments of the present invention relate to kits comprising a metabolic precursor and an imaging probe, more particularly an imaging probe comprising a detectable label which is a contrast agent used in traditional imaging systems. Such a detectable label can be but is not limited to a label selected from the group consisting of MRI-imageable agents, spin labels, optical labels, ultrasound-responsive agents, X-ray-responsive agents, radionuclides, and FRET-type dyes.

In a particular embodiment of the present invention, use is made of reporter probes. Such a reporter probe can be the substrate of an enzyme, more particularly an enzyme which is not endogenous to the cell, but has been introduced by way of gene therapy or infection with a foreign agent. Non-endogenous as referring to a gene in a cell or tissue herein is used to indicate that the gene is not naturally present and/or expressed in that cell or tissue. Alternatively, such a reporter probe is a molecule which is introduced into the cell by way of a receptor or a pump, which can be endogenous or introduced into the cell by way of gene therapy or infection with a foreign agent. Alternatively, the reporter probe is a molecule which reacts to certain (changing) conditions within a cell or tissue environment.

The invention thus provides probes and kits for of use in medical imaging and therapy. Moreover the present invention relates to methods of imaging and methods of treatment using the two components of the invention. According to a particular embodiment the present invention provides for probes and kits for use in targeted and pre-targeted imaging and therapy. Accordingly, the present invention relates to methods of imaging cells, tissues, organs, foreign components, by use of at least two components which are partners in a bio-orthogonal reaction such as the Staudinger ligation. The two components being a targeting probe, target metabolic precursor or reporter probe on the one hand and an imaging probe on the other hand. Moreover, the present invention relates to methods of prevention or treatment, targeting cells, tissues, organs, foreign components, by use of at least two components which are partners in a bio-orthogonal reaction such as the Staudinger ligation. The two components being a targeting probe, target metabolic precursor, or reporter probe on the one hand and a therapeutic probe on the other hand. The present invention further relates to methods of manufacture of the tools used in imaging and therapy of the present invention.

FIG. 1 shows the Staudinger reaction in scheme 1 and the Staudinger Ligation in scheme 2.

FIG. 2 shows scheme 3: the traceless Staudinger ligation.

FIG. 3 presents a general scheme of the pre-targeting concept.

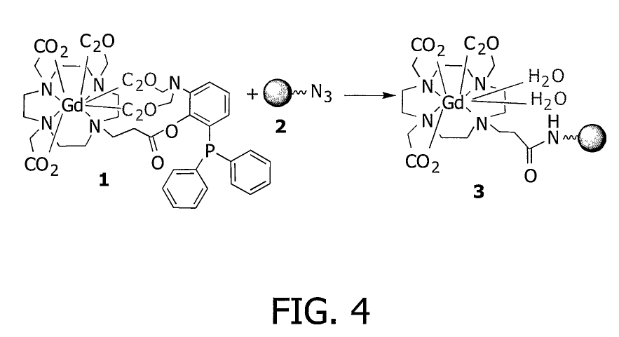

FIG. 4 shows the reaction of an imaging probe comprising a phosphine group and a “smart” MRI contrast agent and an azide group present on a targeting probe bound to its target, whereupon signal intensity is achieved.

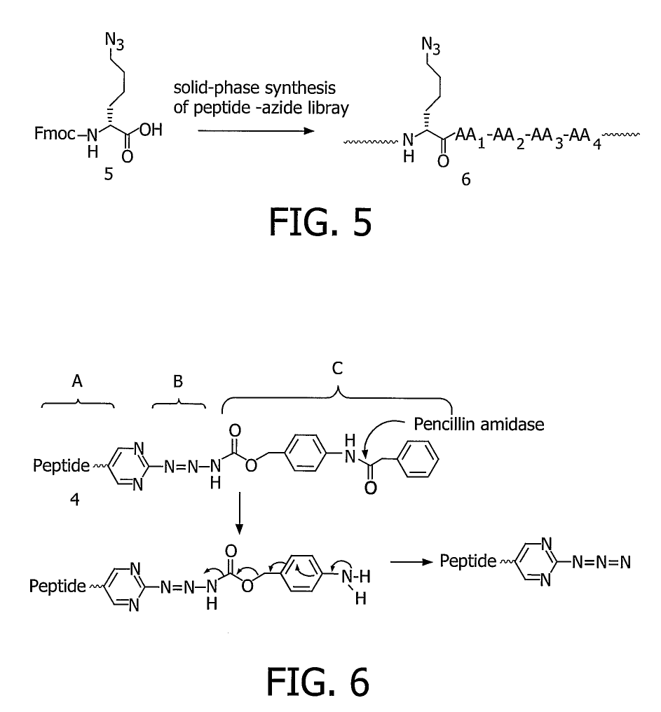

FIG. 5 shows how a binding site for a label or therapeutic compound is included in the design of a combinatorial library for the identification of new leads for a specific target.

FIG. 6 provides an illustration of the imaging of a reporter gene, e.g. a gene encoding penicillin amidase, during gene therapy.

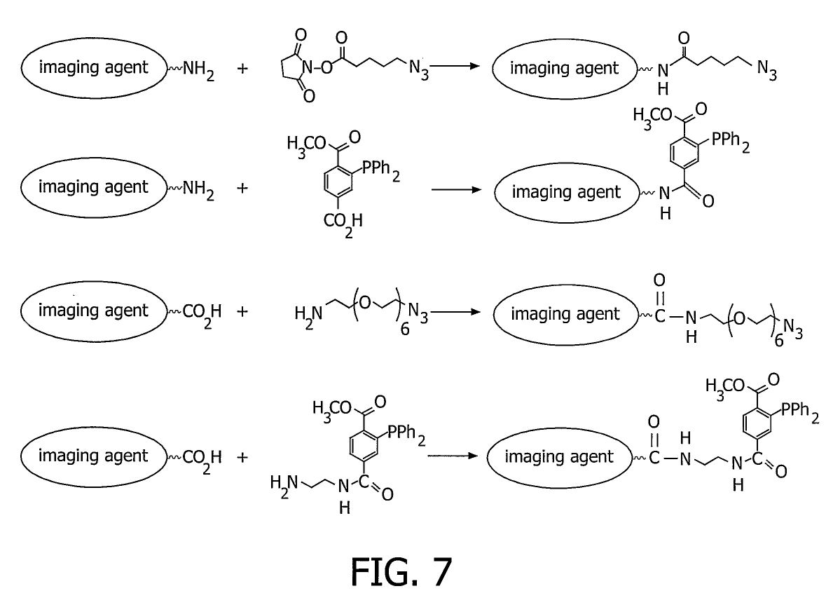

FIG. 7 provides a general synthetic pathway of imaging probes, whereby an imaging agent is conjugated to an azide or phosphine moiety through an amine or carboxylic acid.

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

It is furthermore to be noticed that the term “comprising”, used in the description and in the claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

The present invention provides a solution to the above mentioned limitations of current (pre)targeted imaging, using a covalent ligation, especially a biocompatible covalent ligation instead of biologically based interactions, e.g. the Staudinger ligation, which is a selective chemical and bioorthogonal reaction, [Saxon & Bertozzi (2000) Science 287, 2007-2010; Saxon et al. (2002) J. Am. Chem.Soc. 124, 14893-14902].

The use of a biocompatible direct covalent reaction between two molecules, which does not occur in nature, solves the drawbacks encountered with recognition mechanisms based on non-covalent reactions in different applications. More particularly, it represents a number of advantages of particular interest in pre-targeting and represents a powerful new tool in molecular imaging.

Embodiments of the present invention provide a chemical reaction wherein the two participating functional groups are much smaller than their biological counterparts in current pretargeting combinations. With the methods of the present invention, two participating functional groups, e.g. azide and phosphine, are used which equal the tremendous selectivity of non-covalent recognition events that occur in many biological processes, such as antibody-antigen binding. In accordance with an aspect of the present invention two participating functional groups are selected that have a have finely tuned reactivity so that interference with coexisting functionality is avoided. In accordance with a further aspect of the present invention reactive partners are selected which are abiotic, form a stable adduct under physiological conditions, and recognize only each other while ignoring their cellular/physiological surroundings, i.e. they are bio-orthogonal. The demands on selectivity imposed by a biological environment preclude the use of most other conventional reactions. The Staudinger ligation is a preferred reaction for the methods of the present invention which are performed in a cellular environment. For the methods and compounds of the present invention, both the non-traceless and the traceless Staudinger ligation can be used.

Using the method and compounds of the present invention, imaging probes can be rapidly excreted from the body, due to their small size, e.g. through the kidneys, and can provide the desired high tumor accumulation with relatively low non-target accumulation. In nuclear medical imaging the concept of pre-targeting is advantageous, as the time consuming pre-targeting step can be carried out without using radioactive isotopes, while the secondary targeting step using a radioactive isotope, coupled to a small azide or phosphine comprising the secondary targeting moiety, can be carried out faster. The latter allows the use of shorter-lived radionuclides with the advantage, for example, of minimizing the radiation dose to the patient and allowing the usage of PET i.e. Positron Emission Tomography agents instead of SPECT i.e. Single Photon Emission Computerized Tomography agents. In ultrasound imaging the conventional contrast agents have been based on bubbles and have limited contrast agent lifetime. Pre-targeting concepts according to the present invention can circumvent the problem of limited contrast agent lifetime and make the usage of a universal contrast agent possible. Moreover, the present invention is particularly suitable for use in multimodal imaging, optionally using different imaging agents to visualize the same target.

In accordance with a further aspect of the present invention, a pre-targeting approach is used in combination with multidentate ligand systems such as dendrimers, polymers, or liposomes, so that signal amplification, e.g. MRI signals, at target sites can be accomplished.

The application of the Staudinger Ligation in molecular imaging opens up pre-targeting to all types and sizes of targeting constructs. This allows intracellular and metabolic imaging to profit from the high target accumulation and low background, attainable through pre-targeting build-up. Likewise, pre-targeted signal amplification schemes, e.g. polyazido and/or polyphosphine dendrimers or liposomes, become available for smaller and more diverse targeting devices. As the reaction partners are abiotic and bio-orthogonal, pre-targeting with the Staudinger ligation is not hampered by endogenous competition and metabolism/decomposition, and affords a stable covalent bond. Choosing a target metabolic pathway, and the corresponding azido-metabolite derivative by virtue of its high flux in, for example, tumor cells compared to normal cells, affords the installation of a high density of artificial azido receptors or other chemical handles on the surfaces of target cells, circumventing the use of endogenous cell surface receptors which can sometimes be at low levels.

Finally, the orthogonal nature of the Staudinger ligation makes it the reaction of choice in the generalization of pre-targeting schemes or conjugation chemistry of imaging agents to a wide range of targeting devices and/or with a great variety of functional groups.

The present invention presents novel applications of the Staudinger ligation as one example of a covalent bonding system that is biocompatible and can be used in the human or animal body. It is believed to be bio-orthogonal. The “Staudinger ligation” as referred to in the present application refers to a modification of the Staudinger reaction. In the “Staudinger reaction”, a reaction occurs between a phosphine and an azide to produce an aza-ylide (FIG. 1, Scheme 1). In the presence of water, this intermediate hydrolyzes spontaneously to yield a primary amine and the corresponding phosphine oxide. The phosphine and the azide react with each other rapidly in water at room temperature in a high yield.

In the Staudinger ligation, the Staudinger reaction has been modified to circumvent the hydrolysis of the aza-ylide intermediate. For this purpose, a phosphine bearing an electrophylic trap, e.g. methyl ester, was designed that enables the intramolecular rearrangement of the unstable nucleophilic aza-ylide intermediate into a stable adduct before hydrolysis gets a chance. Thus, the Staudinger ligation proceeds by reaction of this modified and bioconjugated triarylphosphine with an azide conjugate, after which intramolecular cyclization gives an amide bond and phosphine oxide. This ligation is referred to as the “non-traceless” Staudinger ligation (FIG. 1, Scheme 2) as the ligation product contains an appended triphenylphosphine oxide residue. In FIG. 1 the Staudinger reaction is exemplified. The aryl groups specified there are preferred examples but alternatively these may be replaced by any alkyl or cycloalkyl group.

In scheme 2 FIG. 1, R is a) the primary targeting moiety in the case of a targeting probe, b) a detectable label in the case of an imaging probe or c) a therapeutic compound in the case of a therapeutic probe. R′ is a) the primary targeting moiety in the case of a targeting probe, b) a detectable label in the case of an imaging probe or c) a therapeutic compound in the case of a therapeutic probe. In scheme 1 and 2 R′ is linked to one of the aryl groups. It will be appreciated that R′ may also be attached to any other suitable part of the phosphine molecule.

However, a “traceless” Staudinger ligation was later developed to generate a simple amide bond from azide and phosphine reagents (FIG. 2). This reaction utilizes phosphines bearing a transferable acyl group. Reaction with azides generates, after rearrangement of the intermediate aza-ylide and hydrolysis, the amide linked product and a liberated phosphine oxide. In FIG. 2 the aryl groups are preferred examples which may be replaced with any alkyl or cycloalkyl group. In FIG. 2 “X” represents a heterogeneous atom such as oxygen or sulfur. R′ is a) the primary targeting moiety in the case of a targeting probe, b) a detectable label in the case of an imaging probe or c) a therapeutic compound in the case of a therapeutic probe. In the “traceless” Staudinger ligation R′ is attached to the electrophylic trap. R is a) the primary targeting moiety in the case of a targeting probe, b) a detectable label in the case of an imaging probe or c) a therapeutic compound in the case of a therapeutic probe. Use of both the “non-traceless” and the “traceless” Staudinger ligation are envisaged within the context of the present invention.

A “primary target” as used in the present invention relates to a target to be detected by imaging. For example, a primary target can be any molecule which is present in an organism, tissue or cell. Targets for imaging include cell surface targets, e.g. receptors, glycoproteins; structural proteins, e.g. amyloid plaques; intracellular targets, e.g. surfaces of Golgi bodies, surfaces of mitochondria, RNA, DNA, enzymes, components of cell signaling pathways; and/or foreign bodies, e.g. pathogens such as viruses, bacteria, fungi, yeast or parts thereof. Examples of primary targets include compounds such as proteins of which the presence or expression level is correlated with a certain tissue or cell type or of which the expression level is upregulated or downreguated in a certain disorder. According to a particular embodiment of the present invention, the primary target is a protein such as a receptor. Alternatively, the primary target may be a metabolic pathway, which is upregulated during a disease, e.g. infection or cancer, such as DNA synthesis, protein synthesis, membrane synthesis and saccharide uptake. In diseased tissues, above-mentioned markers can differ from healthy tissue and offer unique possibilities for early detection, specific diagnosis and therapy especially targeted therapy.

A “targeting probe” as used herein refers to a probe which binds to the primary target. The targeting probe comprises a “primary targeting moiety” and a “secondary target”.

A “primary targeting moiety” as used in the present invention relates to the part of the targeting probe which binds to a primary target. Particular examples of primary targeting moieties are peptides or proteins which bind to a receptor. Other examples of primary targeting moieties are antibodies or fragments thereof which react with a cellular compound. Antibodies can be raised to non-proteinaceous compounds as well as to proteins or peptides. Other primary targeting moieties can be made up of aptamers, oligopeptides, oligonucleotides, oligosacharides, as well as peptoids and organic drug compounds. A primary targeting moiety preferably binds with high specificity, with a high affinity and the bond with the primary target is preferably stable within the body.

A “secondary target” as used in the present invention relates that part of the targeting probe which provides the reaction partner for the covalent ligation, e.g. the Staudinger ligation which is present on the secondary targeting moiety of the imaging or therapeutic probe described below. In specific embodiments, the secondary target will be one or more azide groups. However, in other particular embodiments, applications are envisaged wherein the secondary target will be one or more phosphine groups.

A “target metabolic precursor” as used herein refers to a substrate of a metabolic pathway which comprises a reaction partner for the covalent ligation, e.g. the Staudinger ligation, i.e. as secondary target, which according to the present invention reacts with the secondary targeting moiety of the imaging or therapeutic probe described below. The metabolic pathway can be a pathway occurring in each cell (like DNA-, protein- and membrane-synthesis) and can be upregulated during for example cancer or inflammation/infection. Alternatively, the metabolic pathway can be specific for a particular cell type, e.g. cancer cells.

The “imaging probe” comprises a “secondary targeting moiety” and a detectable label, such as for instance a contrast providing unit.

A “secondary targeting moiety” relates to the part of the imaging probe comprising a reaction partner for the covalent ligation, e.g. the Staudinger ligation which reacts with secondary target on the primary targeting probe. In particular embodiments the secondary targeting moiety will comprise the one or more phosphine groups.

A “detectable label” as used herein relates to the part of the imaging probe which allows detection of the probe, e.g. when present in a cell, tissue or organism. One type of detectable label envisaged within the context of the present invention is a contrast providing agent. Different types of detectable labels are envisaged within the context of the present invention and are described herein.

A “therapeutic probe” as used herein refers to a probe comprising a secondary targeting moiety and a pharmaceutically active compound, such as but not limited to a therapeutic compound. Examples of pharmaceutically active compounds are provided herein. A therapeutic probe can optionally also comprise a detectable label.

A “combined probe”, i.e. a “combined targeting and imaging probe” or a “combined targeting and therapeutic probe” or a “combined targeting and imaging and therapeutic probe” as used herein refers to the compound resulting from the binding of the secondary target, e.g. an azide or a phosphine, of the targeting probe with the secondary targeting moiety, e.g. a phosphine or an azide, respectively of the imaging probe. This binding can be in vitro. Thus such a combined probe comprises a primary targeting moiety and a detectable label.

The term “isolated” as used herein refers to a compound being present outside the body or outside a cell or fraction of cell, e.g. cell lysate. With respect to particular features attributed to an isolated probe or combined probe, e.g. a primary targeting probe, imaging probe or a therapeutic probe or a combination thereof, in the context of the present invention, this refers to a probe as present outside the human or animal body, tissue or cell. It does not refer to conjugates which are formed within a body, tissue or cell after the consecutive addition of the constituent components of said conjugate to said body tissue or cell.

In a first aspect the invention relates to a method for pre-targeting using compounds which react in covalent ligation, e.g. the Staudinger ligation.

The general concept of pre-targeting is outlined for imaging in FIG. 3. A marker of interest is present on e.g. a cell surface of a certain diseased tissue. This marker is referred to as the “primary target”. In a first pre-targeting step, a targeting probe binds via the primary targeting moiety to the primary target. The targeting probe also carries a secondary target, which will allow specific conjugation to the imaging probe. Optionally, once the targeting probe has reached the primary target and is bound to it, e.g. taking 24 hours to do so, a clearing agent can be used to remove excess targeting probe from the tissue, or organism, if natural clearance is not sufficient. In a second incubation step, e.g. 1-6 hours duration, the imaging probe, which provides the detectable label for the imaging modality, binds to the (pre)-bound targeting probe via its secondary targeting groups.

The advantage of making use of the Staudinger ligation in a pre-targeting strategy is that both phosphine and azide are abiotic and essentially unreactive toward biomolecules inside or on the surfaces of cells and all other regions like serum etc. Thus, the compounds and the method of the invention can be used in a living cell, tissue or organism. Moreover, the azide group is small and can be introduced in biological samples or living organisms without altering the biological size significantly. Using the Staudinger ligation it is possible to bind primary targeting moieties, which are large in size, e.g. antibodies, with labels or other molecules using small reaction partners, e.g. azide and phosphine. Even more advantageously, primary targeting moieties can be bound which are relatively small, eg peptides, with labels or other molecules using (matched) small reaction partners, eg azide and phosphine. The size and properties of the targeting probe and imaging probe are not greatly affected by the secondary target and secondary targeting moiety, allowing (pre)targeting schemes to be used for small targeting moieties. Because of this, other tissues can be targeted, i.e. the destination of the probes is not limited to the vascular system and interstitial space, as is the case for current pretargeting with antibody-streptavidin

According to one embodiment, the invention is used for targeted imaging. According to this embodiment, imaging of specific primary target is achieved by specific binding of the primary targeting moiety of the targeting probe and detection of this binding using detectable labels.

According to the present invention, the primary target can be selected from any suitable targets within the human or animal body or on a pathogen or parasite, e.g. a group comprising cells such as cell membranes and cell walls, receptors such as cell membrane receptors, intracellular structures such as Golgi bodies or mitochondria, enzymes, receptors, DNA, RNA, viruses or viral particles, antibodies, proteins, carbohydrates, monosacharides, polysaccharides, cytokines, hormones, steroids, somatostatin receptor, monoamine oxidase, muscarinic receptors, myocardial sympatic nerve system, leukotriene receptors, e.g. on leukocytes, urokinase plasminogen activator receptor (uPAR), folate receptor, apoptosis marker, (anti-)angiogenesis marker, gastrin receptor, dopaminergic system, serotonergic system, GABAergic system, adrenergic system, cholinergic system, opoid receptors, GPIIb/IIIa receptor and other thrombus related receptors, fibrin, calcitonin receptor, tuftsin receptor, integrin receptor, VEGF/EGF receptors, matrix metalloproteinase (MMP), P/E/L-selectin receptor, LDL receptor, P-glycoprotein, neurotensin receptors, neuropeptide receptors, substance P receptors, NK receptor, CCK receptors, sigma receptors, interleukin receptors, herpes simplex virus tyrosine kinase, human tyrosine kinase.

In order to allow specific targeting of the above-listed primary targets, the primary targeting moiety of the targeting probe can comprise compounds including but not limited to antibodies, antibody fragments, e.g. Fab2, Fab, scFV, polymers (tumor targeting by virtue of EPR effect), proteins, peptides, e.g. octreotide and derivatives, VIP, MSH, LHRH, chemotactic peptides, bombesin, elastin, peptide mimetics, carbohydrates, monosacharides, polysaccharides, viruses, drugs, chemotherapeutic agents, receptor agonists and antagonists, cytokines, hormones, steroids. Examples of organic compounds envisaged within the context of the present invention are, or are derived from, estrogens, e.g. estradiol, androgens, progestins, corticosteroids, paclitaxel, etoposide, doxorubricin, methotrexate, folic acid, and cholesterol.

According to a particular embodiment of the present invention, the primary target is a receptor and suitable primary targeting moieties include but are not limited to, the ligand of such a receptor or a part thereof which still binds to the receptor, e.g. a receptor binding peptide in the case of receptor binding protein ligands.

Other examples of primary targeting moieties of protein nature include interferons, e.g. alpha, beta, and gamma interferon, interleukins, and protein growth factor, such as tumor growth factor, e.g. alpha, beta tumor growth factor, platelet-derived growth factor (PDGF), uPAR targeting protein, apolipoprotein, LDL, annexin V, endostatin, and angiostatin.

Alternative examples of primary targeting moieties include DNA, RNA, PNA and LNA which are e.g. complementary to the primary target.

According to a particular embodiment of the invention, small lipophilic primary targeting moieties are used which can bind to an intracellular primary target.

According to a further particular embodiment of the invention, the primary target and primary targeting moiety are selected so as to result in the specific or increased targeting of a tissue or disease, such as cancer, an inflammation, an infection, a cardiovascular disease, e.g. thrombus, atherosclerotic lesion, hypoxic site, e.g. stroke, tumor, cardiovascular disorder, brain disorder, apoptosis, angiogenesis, an organ, and reporter gene/enzyme. This can be achieved by selecting primary targets with tissue-, cell- or disease-specific expression. For example, membrane folic acid receptors mediate intracellular accumulation of folate and its analogs, such as methotrexate. Expression is limited in normal tissues, but receptors are overexpressed in various tumor cell types.

According to one embodiment, the targeting probe and the imaging probe can be multimeric compounds, comprising a plurality of primary and/or secondary targets and/or secondary targeting moieties. These multimeric compounds can be polymers, dendrimers, liposomes, polymer particles, or other polymeric constructs. Of particular interest for amplifying the signal of detection are targeting probes with more than one secondary target, which allow the binding of several, imaging probes.

According to a particular embodiment of the present invention, the compounds and methods of the present invention are used for imaging, especially medical imaging. In order to identify the primary target, use is made of an imaging probe comprising one or more detectable labels. Particular examples of detectable labels of the imaging probe are contrast agents used in traditional imaging systems such as MRI-imageable agents, spin labels, optical labels, ultrasound-responsive agents, X-ray-responsive agents, radionuclides, (bio)luminescent and FRET-type dyes. Exemplary detectable labels envisaged within the context of the present invention include, and are not necessarily limited to, fluorescent molecules, e.g. autofluorescent molecules, molecules that fluoresce upon contact with a reagent, etc., radioactive labels; biotin, e.g., to be detected through reaction of biotin and avidin; fluorescent tags, imaging agents for MRI comprising paramagnetic metal, imaging reagents, e.g., those described in U.S. Pat. Nos. 4,741,900 and 5,326,856) and the like.

The radionuclide used for imaging can be, for example, an isotope selected from the group consisting of ³H, ¹¹C, ¹³N, ¹⁵O, ¹⁸F, ⁵¹Cr, ⁵²Fe, ^{52m}Mn, ⁵⁵Co, ⁶⁰Cu, ⁶¹Cu, ⁶²Zn, ⁶²Cu, ⁶³Zn, ⁶⁴Cu, ⁶⁶Ga, ⁶⁷Ga, ⁶⁸Ga, ⁷⁰As, ⁷¹As, ⁷²As, ⁷⁴As, ⁷⁵Se, ⁷⁵Br, ⁷⁶Br, ⁷⁷Br, ^{80m}Br, ^{82m}Br, ⁸²Rb, ⁸⁶Y, ⁸⁸Y, ⁸⁹Sr, ⁸⁹Zr, ⁹⁷Ru, ^{99m}Tc, ¹¹⁰In, ¹¹¹In, ^{113m}In, ^{114m}In, ^{117m}Sn, ¹²⁰I, ¹²²Xe, ¹²³I, ¹²⁴I, ¹²⁵I, ¹⁶⁶Ho, ¹⁶⁷Tm, ¹⁶⁹Yb, ^{193m}Pt, ^{195m}Pt, ²⁰¹Tl, ²⁰³Pb. Other elements and isotopes, such as being used for therapy may also be applied for imaging in certain applications.

The MRI-imageable agent can be a paramagnetic ion or a superparamagnetic particle. The paramagnetic ion can be an element selected from the group consisting of Gd, Fe, Mn, Cr, Co, Ni, Cu, Pr, Nd, Yb, Tb, Dy, Ho, Er, Sm, Eu, Ti, Pa, La, Sc, V, Mo, Ru, Ce, Dy, Tl. A particular embodiment of the present invention relates to the use of “smart” or “responsive” MRI contrast agents, as described more in detail hereafter.

The ultrasound responsive agent can comprise a microbubble, the shell of which consisting of a phospholipid, and/or (biodegradable) polymer, and/or human serum albumin. The microbubble can be filled with fluorinated gasses or liquids.

The X-ray-responsive agents include but are not limited to Iodine, Barium, Barium sulfate, Gastrografin or can comprise a vesicle, liposome or polymer capsule filled with Iodine compounds and/or barium sulfate.

Moreover, detectable labels envisaged within the context of the present invention also include peptides or polypeptides that can be detected by antibody binding, e.g., by binding of a detectable labeled antibody or by detection of bound antibody through a sandwich-type assay.

In one embodiment the detectable labels are small size organic PET and SPECT labels, such as ¹⁸F, ¹¹C or ¹²³I. Due to their small size, organic PET or SPECT labels, e.g. ¹⁸F, ¹¹C, or ¹²³I, are ideally suited for monitoring intracellular events as they do not greatly affect the properties of the targeting device in general and its membrane transport in particular. Likewise, the azide moiety is small and can be used as label for intracellular imaging of proteins, mRNA, signaling pathways etc. An imaging probe comprising a PET label and triphenylphosphine as a secondary targeting moiety is lipophilic and able to passively diffuse in and out of cells until it finds its binding partner. Moreover, both components do not preclude crossing of the blood brain barrier and thus allow imaging of regions in the brain.

According to another embodiment the compounds and methods of the invention are used for targeted therapy. This is achieved by making use of a therapeutic probe which comprises a secondary targeting moiety and one or more pharmaceutically active agents (i.e. a drug or a radioactive isotope for radiation therapy). Suitable drugs for use in the context of targeted drug delivery are known in the art. Optionally, the therapeutic probe can also comprise a detectable label, such as one or more imaging agents. A radionuclide used for therapy can be an isotope selected from the group consisting of ²⁴Na, ³²P, ³³P, ⁴⁷Sc, ⁵⁹Fe, ⁶⁷Cu, ⁷⁶As, ⁷⁷As, ⁸⁰Br, ⁸²Br, ⁸⁹Sr, ⁹⁰Nb, ⁹⁰Y, ¹⁰³Ru, ¹⁰⁵Rh, ¹⁰⁹Pd, ¹¹¹Ag, ¹²¹Sn, ¹²⁷Te, ¹³¹I, ¹⁴⁰La, ¹⁴¹Ce, ¹⁴²Pr, ¹⁴³Pr, ¹⁴⁴Pr, ¹⁴⁹Pm, ¹⁴⁹Tb, ¹⁵¹Pm, ¹⁵³Sm, ¹⁵⁹Gd, ¹⁶¹Tb, ¹⁶⁵Dy, ¹⁶⁶Dy, ¹⁶⁶Ho, ¹⁶⁹Er, ¹⁷²Tm, ¹⁷⁵Yb, ¹⁷⁷Lu, ¹⁸⁶Re, ¹⁸⁸Re, ¹⁹⁸Au, ¹⁹⁹Au, ²¹¹At, ²¹¹Bi, ²¹²Bi, ²¹²Pb, ²¹³Bi, ^{214 Bi,} ²²³Ra, ²²⁵Ac.

A number of therapeutic compounds presently known already contain an azide, and thus represent useful therapeutic probes which can be used in combination with a targeting probe, for targeted therapy to improve their efficiency. For instance azide-dye conjugates for photodynamic cancer therapy (see WO 03/003806) can be more efficiently targeted to diseased tissue using a pre-targeting strategy.

In yet another embodiment of the present invention, the use of a targeting probe is replaced by selectively incorporating the secondary target of the invention into a target cell or tissue. This is achieved by using molecules that are involved in pathways in the cell such as metabolic precursor molecules, comprising a secondary target, e.g. an azide reaction partner, that can be incorporated into biomolecules by the metabolism of the cell. Molecules that are involved in pathways in the cells are also referred to as building blocks. The metabolic pathways targeted in this way can be pathways that are common to all cells, such as DNA-, protein- and membrane synthesis. Optionally, these are metabolic pathways which are upregulated in disease conditions such as cancer or inflammation/infection. Alternatively, the targeted metabolic pathways are specific for a particular type of cell or tissue. The target metabolic precursors which can be used in the context of the present invention, include metabolic precursor molecules such as, but not limited to amino acids and nucleic acids, amino sugars, lipids, fatty acids and choline. Imaging of these compounds, such as amino acids, can reflect differences in amino acid uptake and/or in protein synthesis. A variety of sugars can be used for the labelling of carbohydrate structure. Fatty acids can be used for the labelling of lipids in e.g. cellular membranes.

Moreover, a number of analogs of metabolic precursors are known in the art, which can provide particular advantages for use in the context of the present invention. A non-limiting list of examples of metabolic pathways and corresponding metabolic precursors which can be labelled with azide or phosphine are provided below. Some of these become temporarily accumulated into the cell, while others are incorporated into biological macromolecules.

Thymidine phosphorylase is an enzyme that catalyzes the hydrolysis of thymidine to thymine and deoxyribose-1-phosphate. High levels of expression of thymidine phosphorylase have reportedly been associated with decreased survival in colorectal, head or neck, bladder, and cervical cancer and also with angiogenic activity of tumors. Since the enzyme also catalyzes the reverse reaction (i.e., conversion of thymine to thymidine), it can serve as a means of intracellular trapping of therapeutic analogs of thymine, such as capecitabine, which is converted to fluorouracil.

Proliferation-targeted imaging agents preferably have a high specificity for malignant tumors and can be used to differentiate benign or low-grade tumors from high-grade lesions, to detect high-grade transformation in a low-grade tumor, or to plan the optimal approach for diagnostic biopsy, surgical resection, or radiation therapy. FDG and methionine are already in use for this purpose. Most research focuses on DNA analogs, which are incorporated into the replicated DNA strand, such as thymidine or bromodeoxyuridine (BrdU). Bromo-2′-fluoro-2′-deoxyuridine (BFU) is an analog which is more resistant to degradation and has a high incorporation rate. Short plasma half-life can be improved by using cimetidine to inhibit renal elimination of the agent. Other suitable nucleoside analogs include 1′-fluoro-5-(C-methyl)-1-beta-D-arabinofuranosyluracil (FMAU), deoxyuridine; and 5-iodo-1-(2-fluoro-2-deoxy-beta-D-arabinofuranosyl)-uracil (FIAU).

A particular embodiment of the invention relates to the use of reporter probes, i.e. molecules which by their involvement in a cellular process, allow the visualization of a process or cell-type. Such a probe can make use of an endogenous mechanism of the cell, e.g. an endogenous enzyme for which a substrate is provided. Alternatively, such a probe functions by virtue of a foreign gene, referred to as a reporter gene. The reporter gene product can be an enzyme that converts a reporter probe to a metabolite that is selectively trapped within the cell. Alternatively, the reporter gene can encode a receptor or transporter or pump, which results in accumulation of the probe into the cells.

Fluorothymidine is a thymidine analog that is phosphorylated by thymidine kinase-1 (TK1), which can be used as a reporter gene, which results in cellular trapping. In cell culture, uptake correlates with TK1 activity and cellular proliferation.

According to a further embodiment of the invention, the reporter probe is a molecule which responds to a particular environment in a cell or tissue. Tissue hypoxia is central to the pathogenesis of cerebrovascular disease, ischemic heart disease, peripheral vascular disease, and inflammatory arthritis. It is also an ubiquitous feature of the growth of malignant solid tumors, where it bears a positive relationship to the aggressiveness of a tumor, and correlates negatively with the likelihood of response to chemotherapy or radiation therapy. Recent work has suggested that there is a common pathway of response to hypoxia in each of these settings. 2-Nitroimidazole compounds are reduced and trapped in hypoxic cells and can be used as sensors of oxygen tension in ischemic myocardium and tumors. Examples include fluoromisonidazole, fluoroerythronitroimidazole, azomycin-arabinoside, vinylmisonidazole, RP-170 (1-[2-hydroxy-1-(hydroxymethyl)-ethoxy]methyl-2-nitroimidazole) and SR 4554 (N-(2-hydroxy-3,3,3-trifluoropropyl)-2-(2-nitro-1-imidazolyl)acetamide). HL91 is a non-nitroimidazole compound that has a tumoral uptake. Another suitable compound is diacetyl-bis(N4-methylthiosemicarbazone)-copper(II) (ATSM).

Optionally the targeting probe or metabolic precursor or building block or reporter probe already comprises a detectable label. Preferably this label is different from the label that may be introduced in a next step using the Staudinger ligation. Combination of two imaging labels has as potential advantages better target localization, artifact elimination, deliniation of non-relevant (clearance) pathways.

According to the present invention either the targeting probe or the target metabolic precursor molecule on the one hand or the imaging probe or therapeutic probe on the other hand, can include a phosphine or an azide group, as the secondary target or the secondary targeting moiety, respectively, which allow the binding of these probes by the Staudinger ligation.

According to an embodiment of the present invention, the phosphine can be represented by the general structure:

Y-Z-PR₂R₃

wherein Z is selected from alkyl, cycloalkyl and aryl groups substituted with R1 and preferably an aryl group substituted with R₁, wherein R₁ is preferably in the ortho position on the aryl ring relative to the PR₂R₃;and wherein R₁ is an electrophilic group to trap, e.g., stabilize, an aza-ylide group, including, but not necessarily limited to, a carboxylic acid, an ester, e.g., an alkyl ester such as a lower alkyl ester, e.g. an alkyl having 1 to 4 carbon atoms, benzyl ester, aryl ester, substituted aryl ester, aldehyde, amide, e.g. an alkyl amide such as lower alkyl amide, e.g. an alkyl amide having 1 to 4 carbon atoms, aryl amide, an alkyl halide such as a lower alkyl halide, e.g. an alkyl halide having 1 to 4 carbon atoms, thioester, sulfonyl ester, an alkyl ketone such as a lower alkyl ketone e.g. an alkyl ketone having 1 to 4 carbon atoms, aryl ketone, substituted aryl ketone, halosulfonyl, nitrile, nitro and the like;

R₂ and R₃ are generally aryl groups, including substituted aryl groups, or cycloalkyl groups, e.g., cyclohexyl groups where R₂ and R₃ may be the same or different, preferably the same; and

Y corresponds to one of a) the primary targeting moiety in the case of a targeting probe, b) a detectable label in the case of an imaging probe, or c) a therapeutic compound in the case of a therapeutic probe. Y can be linked to the phosphine at a hydrogen or another reactive group at any position on the aryl group Z, e.g., para, meta, ortho; exemplary reactive groups include, but are not necessarily limited to, carboxyl, amine, e.g., alkyl amine such as a lower alkyl amine, e.g. comprising 1 to 4 carbon atoms, aryl amine, ester, e.g., alkyl ester such as a lower alkyl ester, e.g. comprising 1 to 4 carbon atoms, benzyl ester, aryl ester, substituted aryl ester, thioester, sulfonyl halide, alcohol, thiol, succinimidyl ester, isothiocyanate, iodoacetamide, maleimide, hydrazine, and the like. Alternatively, Y may be linked to the phosphine component through a linker. In FIGS. 1 and 2 Y is represented by R′.

Y may be linked to Z or to any other suitable part of the phosphine.

Alternatively, the phosphine present on the targeting or imaging probe has a structure modified to comprise a cleavable linker and is of the general formula:

Y—X₁—R₄—PR₂R₃

wherein X₁ is an electrophile, preferably carbonyl or thiocarbonyl which acts as a cleavable linker; and generally between X1 and R4 a heterogeneous atom such as oxygen or sulfur is present.

R₄ is a linker to the electrophile, and may be an alkyl or a substituted or unsubstituted aryl group; and

Y, R₂ and R₃ are as described above.

Such a phosphine group will react with an azide in a ‘traceless’ Staudinger ligation. In the traceless Staudinger ligation Y is linked to the electrophylic trap.

Examples of R₄—PR₂R₃ are disclosed in US application 2003/0199084. This application also discloses several synthesis methods to prepare phosphine derivatives and is incorporated by reference.

Molecules comprising an azide and suitable for use in the present invention, as well as methods for producing azide-comprising molecules suitable for use in the present invention are known in the art.

A general scheme of synthetic pathways for the production of imaging probes whereby an imaging agent comprising an amine or carboxylic acid is linked to a phosphine or azide moiety is provided in FIG. 7. A similar synthetic pathway is applicable for the production of targeting probes, therapeutic probes, or target metabolic precursors starting from appropriate targeting moieties, pharmaceutical compounds or metabolic precursors respectively, bearing an amine or carboxylic group.

According to another embodiment of the invention, a therapeutic probe is used in combination with an imaging probe. In this embodiment, the therapeutic probe, comprising a therapeutic compound and a secondary targeting moiety is administered directly, e.g. without a targeting probe, and the secondary targeting moiety is used for detection with an imaging probe. Thus, according to this embodiment, the secondary targeting moiety of the therapeutic probe, which in fact functions as a secondary target, and the secondary targeting moiety of the imaging probe are partners in the Staudinger ligation. This embodiment is of use, for instance, in AZT (Azidothymidine) therapy planning and monitoring. AZT (1) is an anti-retroviral drug and the first antiviral treatment to be approved for use against HIV. It already has an azide installed on the sugar moiety. This azide can be used as a handle to bind a labelled Staudinger phosphine probe, allowing AZT imaging in a patient.

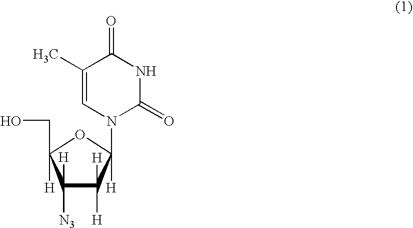

The azide- or phosphine-comprising targeting, imaging and therapeutic probes of the present invention are biocompatible and can be administered in an identical or similar way as conventional molecules which are currently used in medical imaging or therapy. In addition, the detectable labels are known to the skilled person and require conventional methodology and apparatus.

According to a particular embodiment of the invention, the compounds and methods described herein are used in vivo for the imaging or detection of tissues or cell types in the animal or human body. Alternatively, they can be equally used in vitro for the examination of biopsies or other body samples or for the examination of tissues which have been removed after surgery.

As described herein, according to a particular embodiment of the present invention, the targeting probe and imaging or therapeutic probe are provided sequentially, allowing the binding of the targeting probe to its primary target and optionally removal of the excess targeting probe before providing the label or therapeutic compound. This ensures a higher signal to noise ratio in the image and/or a higher efficiency of the therapeutic and is generally referred to as ‘pre-targeting’ or ‘two-step’ targeting. The compounds of the present invention allow a two-step targeting method wherein the problems (excessive long diffusion to the target and clearance from the organism, decay of imaging compound) traditionally related to the size of the secondary target and secondary targeting moieties (ensuring the recognition and binding between the two steps) are circumvented. Moreover such a ‘two-step’ targeting allows the development of ‘universal’ imaging probes, which can be used in combination with the ‘targeting probe’ of interest.

According to a further embodiment the methods and compounds of the present invention are used for targeted signal amplification and/or polyvalency installation. Herein a primary targeting moiety of the targeting probe is conjugated to a dendrimer, polymer or liposome containing multiple triphenylphosphine moieties. After receptor binding of the targeting probe through its primary targeting moiety, an imaging probe comprising an azide conjugated to one or more MRI contrast agents, e.g. Gd chelates, or to an ultrasound reporter, e.g. microbubble, is injected. Herein said contrast agents or microbubbles comprise secondary targeting moieties. The subsequent Staudinger ligation results in a high concentration of MRI contrast agent at the target tissue. Furthermore, the polyvalency at the target site will increase the reaction kinetics with the azide reporter conjugate (imaging probe), affording an efficient target accumulation of MRI contrast agent or microbubbles.

Alternatively, the azide can also be comprised in the targeting probe as mentioned above and the triphenylphosphine conjugated to the reporter in the imaging probe.

According to another aspect the methods, compounds of the invention are used without primary targeting moiety, but incorporate the secondary target into a precursor molecule to be incorporated into biomolecules by the metabolism of the cell. In this way, general metabolic pathways can be targeted. The above-described phosphines or azides are linked e.g. to sugars, amino acids or nucleotides, which can then be administered to the cell or organism and are incorporated into biomolecules and/or trapped in the cell by the normal metabolism. Examples of such incorporation into living organisms, eukaryotic cultivated cells or recombinant protein expression systems (bacteria, yeasts, higher eukaryotes) are described in the art [Lemieux et al 2003 cited above, Hang et al. (2003) Proc Natl. Acad Sci. USA 100, 14846-14851; Wang et al. (2003) Bioconjugate Chem. 14, 697-701].

In a particular embodiment of this aspect of the invention a metabolic pathway, which is upregulated during a disease, like infection/inflammation or cancer, is targeted. Components which can be upregulated in disease conditions include for example DNA, protein, membrane synthesis and sacharide uptake. Suitable building blocks to target these pathways include azide-labeled amino acids, sugars, nucleobases and choline and acetate. These azide labeled building blocks are funtionally analogous to the currently used metabolic tracers [¹¹C]-methionine, [¹⁸F]-fluorodeoxyglucose (FDG), deoxy-[¹⁸F]-fluorothymidine (FLT), [¹¹C]-acetate and [¹¹C]-choline. Cells with a high metabolism or proliferation have a higher uptake of these building blocks. Azide-derivatives can enter these pathways and accumulate in and/or on cells. After sufficient build-up and clearance of free building block, an imaging probe, e.g. a probe comprising a radioactive label and a (cell permeable) Staudinger phosphine as a secondary targeting moiety, is sent in to bind the accumulated azide metabolite. The advantage over normal FDG-type imaging is that there is ample time to allow high build up of the targeting probe before radioactivity is allowed to bind, thus increasing the signal to noise ratio. Alternatively, a metabolic pathway and/or metabolite that is specific for a disease could be targeted.

Another aspect of the invention relates to the use of “smart” or responsive MRI contrast agents in targeted imaging based upon the traceless Staudinger ligation.

According to this aspect of the invention, imaging probes comprising a phosphine group and a “smart” MRI contrast agent having low relaxivity and consequently low signal intensity, achieve a signal intensity upon reacting with an azide group present on a targeting probe. Such “smart” MRI imaging probes comprising a phosphine group, e.g. as secondary targeting moiety, have the general structure (2) as shown hereafter:

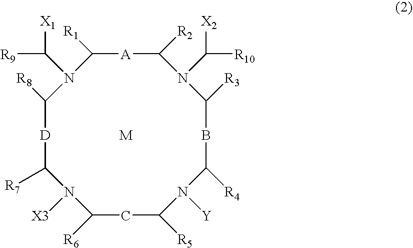

wherein M is a paramagnetic metal ion selected from the group consisting of Gd, Fe, Mn, Cr, Co, Ni, Cu, Pr, Nd, Yb, Tb, Dy, Ho, Er, Sm, Eu, Ti, Pa, La, Sc, V, Mo, Ru, Ce, Dy, Tl;

A, B, C and D are either single bonds or double bonds; X₁, X₂, and X₃ are —OH, —COO—, —CH₂OH—, CH₂COO—, C(O)N—;

R₁-R₁₀ are hydrogen, alkyl, aryl, phosphorus moiety,

and wherein Y is the secondary targeting moiety, more particularly a substituted triphenyl phosphine such as (CH₂)₂—COO—C₆H₄N(CH₂COOH)₂—P—(C₆H₅)₂ or another substituted triphenyl phosphine which allows one or more coordinations between the substituted phosphine and the paramagnetic metal.

An embodiment of this aspect is shown in FIG. 4. A targeting probe comprising an azide group as secondary target is allowed to bind its target (2). Subsequently, the imaging probe consisting of a Gd-DOTA triphenylphosphine conjugate (1) is administered. In this conjugate, the two highlighted carboxylic acids block two water coordination places in the inner sphere of the Gd chelate complex, which leads to a low relaxivity of the construct. This can result in a low signal intensity in MRI. When the azide group of the targeting probe reacts with the triphenylphosphine moiety of the imaging probe, the traceless ligation results in the elimination of the appended carboxylic acids while conjugating the activated MRI probe to the target (3). The binding event thus makes two coordination places available for water, which leads to a signal increase upon binding. This allows MRI imaging of markers within a tissue, as component 1 and 2 diffuse fast within the interstitial space.

Yet another aspect of the invention relates to the provision of a combined targeting and imaging or therapeutic probe, for use in imaging and therapy. Thus, according to this aspect the secondary target of the targeting probe and the secondary targeting moiety of the imaging probe or therapeutic probe are allowed to react in vitro, before administration to the cells, tissue or organism. When linking the targeting probe and the imaging or therapeutic probe of the present invention using the Staudinger ligation, most particularly the traceless Staudinger ligation, there is hardly any increase in size, which is a typical feature of the chemical reaction itself. As a result, the combined targeting probe and imaging probe can be small enough to allow a quick diffusion to the target and a quick clearance from the body. Thus, the Staudinger ligation is envisioned as an orthogonal and general route for the conjugation of imaging agents to targeting constructs. In general, combinations of all of the above-mentioned primary targeting moieties and detectable labels can be produced in vitro and used for this application. It will be understood that for optimal use in vivo, the combined size of the primary targeting moiety and the label should allow sufficiently quick diffusion to the primary target and clearance from the body.

According to a particular embodiment of the above-described aspect of the invention, the combined targeting and imaging/therapeutic probe comprises, as a primary targeting moiety, a peptide which interacts with another protein such as a receptor.

According to another particular embodiment of the above-described aspect of the invention, the detectable label is selected from the group of an organic PET labelled prosthetic group, a metal complex for PET, SPECT, or MRI, a microbubble for ultrasound imaging, and an iodine or barium-containing molecule or vesicle.

An important advantage in the production of the combined targeting and imaging or therapeutic probe of the present invention, is that the individual reagents of the Staudinger ligation (i.e. the targeting probe comprising the secondary target and the imaging or therapeutic probe comprising the secondary targeting moiety) are stable and that the reaction between them occurs in water.

Thus, imaging probes comprising a pendant azide moiety and the targeting probes comprising a triphenylphosphine derivative, or vice versa can be produced and stored separately and combined either beforehand as part of production or just before use by the end-user. The reaction is rapid and gives high yields, and the combined product does not require elaborate purification.

Thus, the present invention also envisages combined targeting and imaging or therapeutic probes, or a kit comprising one or more targeting probes for combining with one or more imaging or therapeutic probes. According to a particular embodiment the two components are reacted in vitro, for example just before the imaging procedure and the combined probe is used as such. According to an embodiment, the combined probe is characterized by the presence of an amide bond which is the result of the reaction of the phosphine and the azide in the traceless Staudinger ligation. Using a non-traceless ligation, the combined probe comprising the primary targeting moiety and the detectable label will comprise a phosphine oxide. For example the primary targeting moiety and the detectable label can be bound to substituents on a phenylgroup of triphenylphosphine. Staudinger kit applications are especially envisaged for nuclear imaging agents.

In a further aspect the invention relates to a pharmaceutical composition comprising the imaging probe according to the invention.

The invention further relates to a method for the preparation of a diagnostic composition for imaging, comprising using the imaging probe according to the invention.

Another aspect of the invention relates to the production of suitable targeting probes for use in the context of the present invention using combinatorial peptide synthesis. In case of peptides, labeling with an azide or tri-phenylphosphin group can disturb the receptor affinity properties. Consequently, after labeling of the targeting moiety, additional time-consuming optimization rounds may be necessary to ensure that the targeting probe has the desired pharmacological properties and receptor affinity. According to this aspect of the present invention, the azide or triphenylphosphin is included in the design of a combinatorial library for the identification of new leads for a specific target. The leads generated by such a methodology do not require additional modification but can be directly used as targeting probes in the context of the present invention. According to a particular embodiment, an amino acid building block carrying an azide residue (5) (FIG. 5) is incorporated at any desired position in the peptide chain during combinatorial preparation of a peptide library (6). Peptide-azide probes are screened for optimal receptor binding affinity. The optimal peptide azide probes can thus be identified for use in pre-targeting.

Another aspect of the invention relates to the use of the Staudinger ligation in the preparation of targeted imaging or therapeutic agents, corresponding to the combined probes of described herein.

Generally, targeted imaging agents are developed by labeling a known targeting group, such as a receptor-binding moiety, e.g. a bioactive peptide or an organic drug-like structure, with a label, such as a radioactive isotope, a metal chelate or an organic fluorophore. The nuclear isotopes are usually bound via a chelate group, e.g. SPECT agents, or an aromatic prosthetic group for halogen labeling. Similarly, targeted therapeutic compounds are developed by linking a known targeting group to a therapeutic compound. These added labeling groups or therapeutic compounds can significantly alter the properties and receptor affinity of the targeting moiety, leading to a sub-optimal targeted imaging agent or therapeutic. Consequently, for each labeled targeting moiety, additional time-consuming optimization rounds of the conjugate with respect to pharmacological properties and target affinity are necessary. According to the present invention, the Staudinger ligation can be used to facilitate the development of such targeted imaging or therapeutic agents. Selective incorporation of the azide or triphenylphosphin at different positions is included in the synthesis of the peptide acting as targeting moiety. This optimization can be applied for the development of targeted probes or therapeutics based on known peptide moieties, i.e. incorporating the azide or triphenylphosphin at different positions in the known peptide sequence. Alternatively, this can be applied in the development of new targeting moieties, e.g. by inclusion in the design of the combinatorial library of new leads for a specific target. The combinatorial library is then bound to the desired label or therapeutic using the Staudinger ligation and screened for both optimal binding affinity to the target and label/therapeutic efficiency.

Optionally, a Staudinger phosphine linked to a cold isotope, e.g. non-radioactive F, I, etc, is coupled to all azide-functionalized library members for studying binding affinities. The “hot” analog can then be obtained by coupling of the azide-peptide to the Staudinger phosphine labelled with the corresponding hot isotope.

The leads generated by such a methodology do not require additional modification but can, after binding of the label or therapeutic compound to the provided binding site, be directly applied as imaging and/or therapeutic agents.

According to a particular embodiment, an amino acid building block carrying an azide residue (5) (FIG. 5) is incorporated at any desired position in the peptide chain during combinatorial preparation of a peptide library (6). Peptide-azide-label or peptide-azide-theraputic conjugates with optimal receptor binding affinity can then be identified for use in targeted imaging or targeted therapeutics, respectively. The combinatorial library thus obtained can comprise an array of a prior identified lead molecule in which the amino acid building block carrying an azide residue is introduced in different positions, to identify by a screening the molecule which displays minimal interaction of the label/therapeutic with the binding of the lead to its target.

The probes and kits of the present invention are of use in medical imaging and therapy, more particularly ‘targeted’ imaging and therapy. The term ‘targeted’ relates to the fact that the imaging label or pharmaceutically active compound upon administration to the patient specifically interacts with or introduced into a target molecule. This can be achieved according to the present invention by use of a targeting probe comprising a targeting moiety or by use of a target metabolic substrate. Alternatively this can be obtained by providing a combined targeting and imaging or therapeutic probe (i.e. administration of the two components of the present invention as a combined probe). This target molecule can be specific for a particular type of cell or tissue or can be common to all cells or tissues in the body. A particular aspect of the present invention relates to ‘pretargeted’ imaging or therapy. This aspect requires the separate use of the two components of the present invention and relates to the separation in time of the administration to the patient of the component which comprises the targeting moiety or ensures the targeting by being a substrate of a particular reaction and the component which ensures the image or therapeutic effect. The time in between administration of the two components can vary but ranges from about 10 minutes to several hours or even days.

The probes of the invention can be administered via different routes including intravenous injection, oral administration, rectal administration and inhalation. Formulations suitable for these different types of administrations are known to the skilled person.

Therapeutic probes or imaging probes comprising a pharmaceutical composition according to the invention can be administered together with a pharmaceutically acceptable carrier. A suitable pharmaceutical carrier as used herein relates to a carrier suitable for medical or veterinary purposes, not being toxic or otherwise unacceptable. Such carriers are well known in the art and include saline, buffered saline, dextrose, water, glycerol, ethanol, and combinations thereof. The formulation should suit the mode of administration.

### EXAMPLES

### Example 1

### Pre-Targeted Imaging of Neuroendocrine Tumours

A targeting probe comprising a somatostatin receptor-binding peptide; e.g. representing a primary targeting moiety in accordance with FIG. 3, linked to an azide group, e.g. as a secondary target, is injected into a subject. After binding of the targeting probe to the primary target, e.g. the somatostatin receptor, present for example in high concentration on neuroendocrine tumours, and clearance of unbound targeting probe, an imaging probe comprising a ¹⁸F-label, i.e. radioactive linked to a Staudinger phosphine group, which acts as secondary targeting moiety, is injected into the subject, e.g. animal or human; where it binds the immobilized azide. The presence of the neuroendocrine tumor can thus be visualised by the radioactive isotope providing the contrast. Alternatively, the secondary targeting moiety of the imaging probe contains the azide while the triphenylphosphine is the secondary target in the targeting probe. Phosphine-(3) or azide-labeled (4) amino acids can be incorporated in a receptor-binding peptide for the production of the targeting probe.

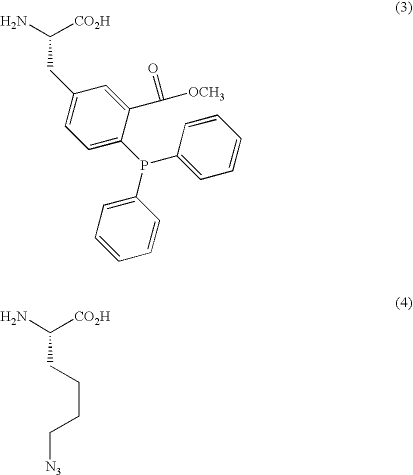

### Example 2

### Pre-Targeted Imaging of Breast Tumor Tissue for Therapy Planning

A targeting probe made up of an azide-estrogen derivative is administered to a breast cancer patient. After estrogen receptor binding, a Staudinger phosphine group conjugated to a ^{99m}Tc chelate is injected as an imaging probe and binds and visualizes the immobilized azide. Several breast cancer-targeted constructs functionalized with an azide are already known. None of these has been used however in a Staudinger Ligation based imaging method.

### Example 3

### Imaging of Bone Tumour Tissue

A conjugate of a diphosphonate with a Staudinger phosphine group is administered as a targeting probe to a bone cancer patient. After bone accumulation, a ^{99m}Tc chelate functionalized with a pendant azide is injected into the patient as the imaging probe. Alternatively, in the context of bone cancer therapy, the imaging probe made up of the chelate-azide conjugate further carries a therapeutic nuclide. Alternatively, in the targeting probe, the diphosphonate is linked to the azide (5) and in the imaging probe, the secondary targeting group is a phosphine group which is linked to the label (Tc chelate).

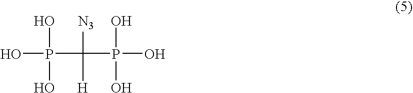

### Example 4

### Pre-Targeted Imaging of Brain Tissue

An azido-tropane derivative is injected as a targeting probe into a subject with e.g. Parkinson disease. After target binding in the dopaminergic system, an ¹⁸F-labelled Staudinger phosphine probe is injected as an imaging probe and binds to the immobilised azide.

### Example 5

### Pre-Targeted Imaging of Hypoxic Tissue

Azide functionalized nitroimidazole derivatives are used as probes to image hypoxia, e.g. (6), (7) shown below. In hypoxic cells the nitro moiety is reduced to a radical, which is then trapped upon reaction with intracellular macromolecules. Subsequently, a lipophilic ¹⁸F-labelled Staudinger phosphine group is injected, e.g. as imaging probe, to bind the accumulated azide.

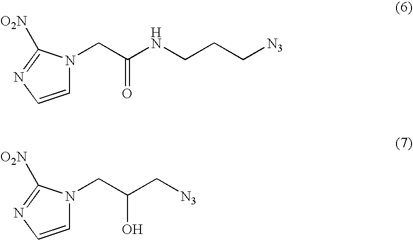

### Example 6

### Pre-Targeted Signal Amplification and/or Polyvalency Installation

A primary targeting moiety is conjugated to a dendrimer or polymer containing multiple triphenylphosphine moieties. After binding of the primary targeting moiety to its primary target, e.g. a receptor, an azide conjugated to one or more MRI contrast agents, e.g. Gd chelates, or to an ultrasound reporter, e.g. microbubbles, is injected as an imaging probe. The subsequent Staudinger ligation results in a high concentration of MRI contrast agent at the target site. Furthermore, the polyvalency at the target site will increase the reaction kinetics with the azide reporter conjugate, affording an efficient target accumulation of MRI contrast agent or microbubbles. Alternatively, the targeting probe comprises the azide in the dendrimer and the triphenylphosphine is conjugated to the label in the imaging probe.

### Example 7

### Pre-Targeting of “Smart” or Responsive MRI Contrast Agents Using the Traceless Staudinger Ligation.

This example is illustrated in FIG. 4. A targeting probe comprising an azide group is allowed to bind its target (2). Subsequently, an imaging probe which is a Gd-DOTA triphenylphosphine conjugate 1 is administered. The two carboxylic acids block two water coordination places in the inner sphere of the Gd chelate complex, which leads to a low relaxivity and hence low signal intensity in MRI of the construct. When the azide group reacts with the triphenylphosphine moiety, the traceless Staudinger ligation results in the elimination of the appended carboxylic acids while conjugating the activated MRI probe to the target (3). The binding event thus makes two coordination places available for water, which leads to a signal increase upon binding. Furthermore, this concept allows MRI imaging of markers within a tissue, as component 1 and 2 will diffuse fast within the interstitial space.

### Example 8

### Imaging a Reporter Gene During Gene Therapy

In this application a vector is used wherein both a therapeutic gene is expressed as well as a reporter gene for the enzyme HSV1-TK. This enzyme metabolically traps uracil analogs and acycloguanosine analogs in the cell. In this embodiment, uracil and acycloguanosine analogs are functionalized with an azide moiety. These molecules are metabolically trapped in tissue where the reporter gene (and thus also the therapeutic gene) is expressed. Subsequently, an ¹⁸F-labelled Staudinger phosphine probe is injected to bind the accumulated azide-comprising uracil and acycloguanosine analogs.

### Example 9

### Imaging a Reporter Gene During Gene Therapy.

This example is illustrated in FIG. 6. A reporter enzyme activates an intracellular delivered azide precursor, which in turn binds to the Staudinger phosphine probe. Masked azide-derivative 4 comprises a transmembrane targeting peptide (A) conjugated to an azide moiety (B). This moiety is protected by an ester group, which is part of an enzyme-sensitive triggering device (C), previously published by Gopin et al. [in (2003) Angew. Chem. Int. Ed., 42, 327-332.] The reporter gene corresponds to the enzyme Penicillin Amidase, which cleaves the benzyl amide moiety in (C). The resulting primary amine effects rearrangement into a methylenecyclohexadienimine and release of the azide-ester. Spontaneous decarboxylation yields the active azide, which can then trap a labelled Staudinger phosphine probe.

### Example 10

### Use of the Staudinger Ligation in the Immobilization of Gd-Dotam Complexes Embedded in the Surface of Liposomes

Complex 8 with its appended stearoyl lipid residue is allowed to cover the surface of a liposome, via insertion of the stearoyl residue in the phospholipid bilayer. Next, the appended azide groups are intermolecularly cross-linked by polyfunctional Staudinger phosphine probe 9 (a tripeptide). The rotation of the cross linked Gd complexes will be dramatically reduced as compared to free Gd complex in the lipid, affording an increased relaxivity in MRI.

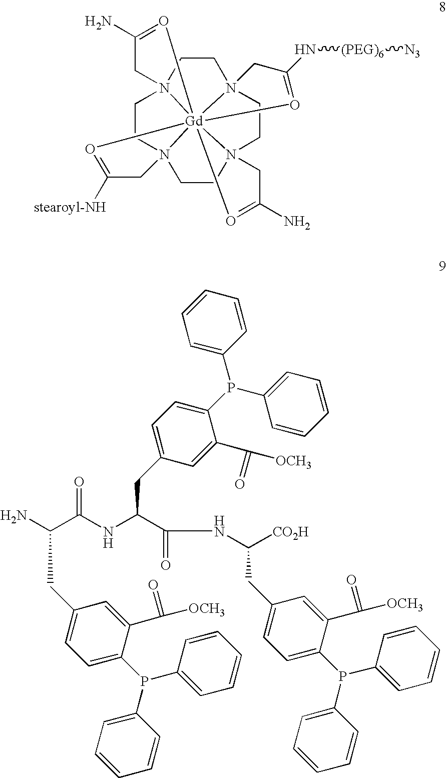

### Example 11

### Synthesis of Triphenylphosphine-DOTA Imaging/Therapeutic Probes 26, 28, and 29

Experimental

The synthesis of the compounds described below was subcontracted to and performed by SyMO-Chem in Eindhoven, The Netherlands.

Instrumentation: NMR spectra were recorded on a Bruker 400 MHz spectrometer, a Varian Gemini 300 MHz spectrometer, and a Varian Mercury 200 MHz spectrometer. Infrared spectra were measured on a Perkin Elmer 1600 FT-IR. MALDI-TOF spectra were obtained at a Perseptive Biosystems Voyager DE-Pro MALDI-TOF mass spectrometer (accelerating voltage: 20 kV; grid voltage: 74.0%, guide wire voltage: 0.030%, delay: 200 ms, low mass gate 900 amu). Samples for MALDI-TOF were prepared by adding a solution of the polymers in THF (20 μl, c=1 mg/ml) to a solution of α-cyano-4-hydroxycinnamic acid in THF (10 μl, c=20 mg/ml) and subsequent thoroughly mixing. This mixture (0.3 μl) was brought on a sample plate, and the solvent was evaporated. Solvents were dried over molsieves prior to use. Hygroscopic compounds were stored in a desiccator over P₂O₅. All reaction were carried out under an argon atmosphere.

The following abbreviations are used: DCM=dichloromethane, DMF=dimethylformamide, DIPEA=diisopropyl ethylamine, TFA=trifluoroacetic acid, HBTU=O-(Benzotriazol-1-yl)-N,N,N′,N′-tetramethyluronium hexafluorophosphate, DCC=N,N′-dicyclohexylcarbodiimide.

Phosphine-Building Blocks:

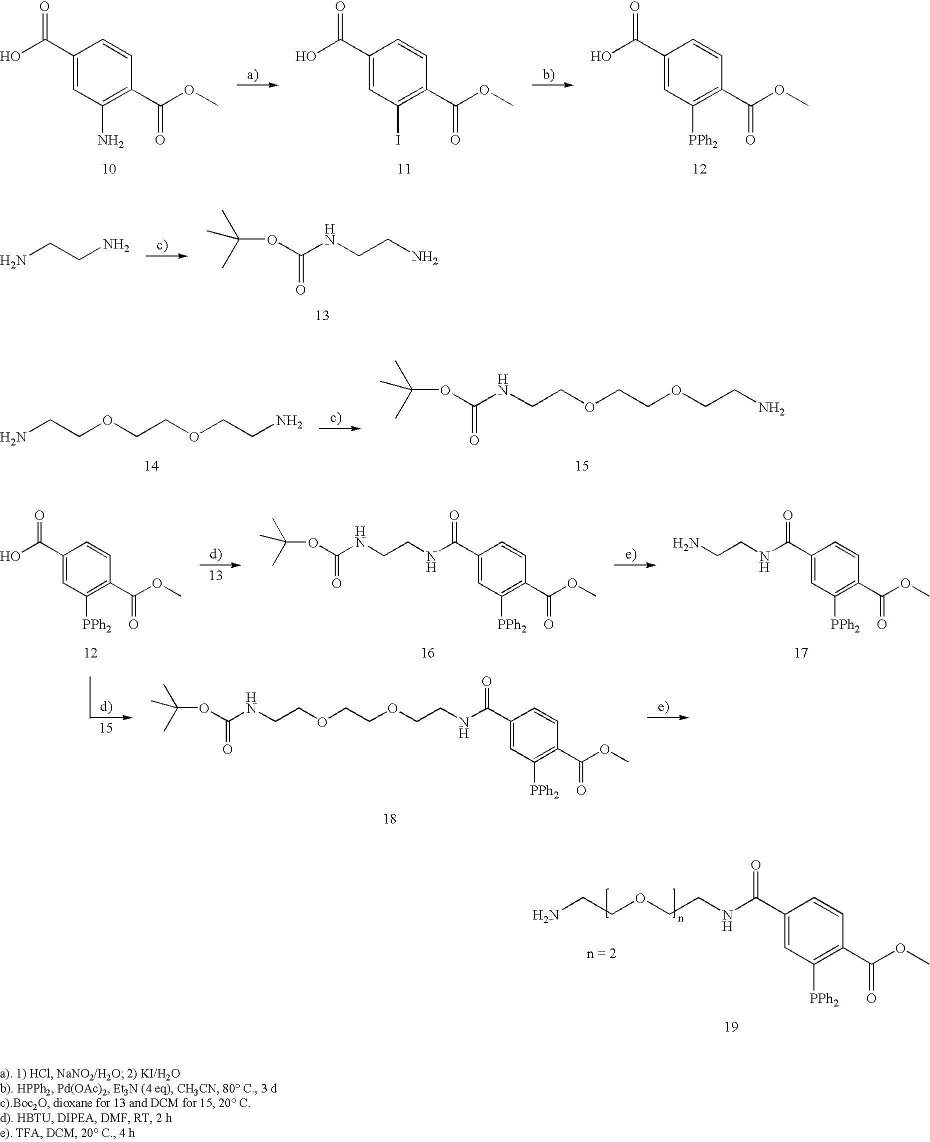

Amino acid 10 is commercially available from Aldrich, just as the diamine 14. The iodide 11 has been prepared according to Saxon et al. US2003199084, while the procedure for the phosphine 12 that has also been reported in this patent has been modified to improve the yield.

Compound 12

To a flame dried flask was added acetonitrile (30 mL), triethylamine (5.32 g; 52.3 mmol), compound 11 (3.06 g; 10.0 mmol) and palladium acetate (42 mg; 0.2 mmol). The mixture was degassed in vacuo. While stirring under an atmosphere of argon, diphenylphosphine (2.16 g; 11.6 mmol) was added to the flask via a syringe. The resulting solution was heated at 80° C. for 3 d, and then allowed to cool to rt and concentrated. The residue was dissolved in DCM (175 mL), washed with H₂O (175 mL), and 1 M hydrochloric acid (2 times 50 mL), and concentrated. The crude product was dissolved in hot MeOH (100 mL) and cooled to 4° C. Filtration afforded the product as a golden yellow solid (2.87 g; 78%), mp=206° C. ¹H-NMR(400 MHz, CDCl₃): δ 10.8 (br.s, 1H), 8.07 (d, 2H, J=1), 7.67 (d, 1H, J=3.4), 7.37-7.25 (m, 10H), 3.75 (s, 3H) ppm. ¹³C-NMR(100 MHz, CDCl₃): δ 170.58, 166.68, 141.46 (d, J=29.5), 138.90 (d, J=19.1), 136.89 (d, 10.0), 135.56, 133.83 (d, J=10.0), 131.90, 130.56, 129.70, 129.02, 128.65 (d, J=7.1), 52.35 ppm. ³¹P-NMR(80 MHz, CDCl₃): δ −4.04 ppm. FT-IR (ATR): ν 2952, 2846, 2536, 1726, 1686, 1434, 1262, 1244, 1106, 1057, 743 cm⁻¹.

N-Mono(tert-butyloxycarbonyl)-ethylenediamine (13)

Di-tert-butyl-dicarbonate (40.77 g; 0.187 mol) was dissolved in dioxane (200 mL) and added dropwise to a solution of ethylenediamine (89.82 g; 1.49 mol) in dioxane (500 mL). After 30 min the suspension was evaporated to dryness and H₂O (500 mL) was added. The white solid was filtered off, and the aqueous layer was extracted with DCM (3 times 250 mL). The combined organic layers were dried with Na₂SO₄ and concentrated. The product was obtained as a colorless liquid (23.75 g; 79%). ¹H-NMR(300 MHz, CDCl₃): δ 5.32 (br.s, 1H), 3.17 (q, 2H, J=6.0), 2.80 (t, 2H, J=6.0), 1.45 (s, 9H), 1.30 (s, 2H) ppm. ¹³C-NMR(75 MHz, CDCl₃): δ 156.09, 78.84, 43.19, 41.65, 28.20 ppm. FT-IR (ATR): ν 3363, 2975, 2932, 1689, 1518, 1364, 1249, 1166, 873 cm⁻¹.

Compound 15

The commercially available diamine 14 (10.7 g; 72.2 mmol) was dissolved in DCM (400 mL) and the solution was heated in an oil bath to 40-45° C. and kept under an argon atmosphere. In three portions di-tert-butyl-dicarbonate (total of 15.9 g; 72.8 mmol) was added and the solution was stirred overnight at 40-45° C. under argon. The solution was cooled down, concentrated in vacuo and the crude product was purified by silica column chromatography using chloroform/MeOH/isopropylamine=20/2/1 to isolate the product as an oil (7.8 g; 44%). ¹H NMR (CDCl₃) δ=5.2 (bs, 1H), 3.5-3.35 (8H), 3.2(q, 2H), 2.75 (t, 2H), 1.35 (s, 9H), 1.25 (b, 2H). ¹³C NMR (CDCl₃) δ=155.7 (C═O), 78.5 (CCH₃), 73.2 and 69.9 (CH₂O), 41.5 and 40.0 (CH₂N), 28.1 (CCH₃).

Compound 16

HBTU (3.64 g; 9.61 mmol) was dissolved in dry DMF (40 mL) and DIPEA (2.48 g; 19.22 mmol) and carboxylic acid 12 (3.50 g; 9.61 mmol) were added. The mixture was stirred under argon at rt for 10 min, and then a solution of amine 13 (1.69 g; 10.57 mmol) in DMF (4 mL) was added. The mixture was stirred for 2 h, subsequently diluted with ether (500 mL), and washed with sat. NaHCO₃ (3 times 400 mL) and 0.1 M HCl (400 mL). The organic layer was dried with Na₂SO₄ and concentrated to yield a yellow solid (4.59 g; 94%). ¹H-NMR(300 MHz, CDCl₃): δ 8.07 (dd, 1H, J=3.3, 7.8), 7.75 (dd, 1H, J=1.8, 8.1), 7.38-7.27 (m, 11H), 6.93 (br.s, 1H), 4.85 (br.s, 1H), 3.75 (s, 3H), 3.44 (q, 2H, J=6.0), 3.31 (q, 2H, J=6.0), 1.42 (s, 9H) ppm. ³¹P-NMR(80 MHz, CDCl₃): δ −3.88 ppm. FT-IR (ATR): ν 3343, 2974, 1716, 1708, 1687, 1637, 1535, 1435, 1274, 1255, 1184, 1165, 1116, 743, 695 cm⁻¹.

Compound 17

Boc-protected amine 16 (4.59 g; 9.06 mmol) was dissolved in DCM (20 mL) and TFA (20 mL), and stirred at rt for 4 h. The mixture was concentrated, redissolved in DCM (100 mL), and washed with sat. Na₂CO₃ (200 mL). The aqueous layer was back extracted with DCM (50 mL), and the combined organic layers were dried with Na₂SO₄ and evaporated to dryness to yield a yellow foam (3.51 g; 97%). ¹H-NMR(200 MHz, CDCl₃): δ 8.08 (dd, 1H, J=3.6, 7.8), 7.81 (dd, 1H, J=1.6, 8.2), 7.37-7.23 (m, 11H), 6.57 (br.s, 1H), 3.72 (s, 3H), 3.33 (q, 2H, J=5.6), 2.79 (t, 2H, J=5.9), 1.14 (br.s, 2H) ppm. ³¹P-NMR(80 MHz, CDCl₃): δ −3.87 ppm. FT-IR (ATR): ν 3301, 2949, 1717, 1641, 1537, 1433, 1271, 1251, 1114, 742, 695 cm⁻¹.

Compound 18

HBTU (2.08 g; 5.49 mmol) was dissolved in dry DMF (25 mL) and DIPEA (1.42 g; 5.49 mmol) and carboxylic acid 12 (2.00 g; 5.49 mmol) were added. The mixture was stirred under argon at rt for 10 min, and a solution of amine 15 (1.50 g; 6.04 mmol) in DMF (2 mL) was added. The mixture was stirred for 2 h, subsequently diluted with ether (300 mL), and washed with sat. NaHCO₃ (3 times 200 mL) and 0.1 M HCl (200 mL). The organic layer was dried with Na₂SO₄ and concentrated to yield a yellow foam (2.90 g; 89%). ¹H-NMR(200 MHz, CDCl₃): δ 8.08 (1H), 7.79 (1H), 7.33-7.27 (11H), 6.41 (1H), 4.94 (1H), 3.74 (3H), 3.59-3.51 (10H), 3.30 (2H), 1.43 (9H) ppm. ³¹P-NMR(80 MHz, CDCl₃): δ −3.82 ppm. FT-IR (ATR): ν 3334, 2896, 1714, 1648, 1525, 1271, 1249, 1166, 1112, 744, 696 cm⁻¹.

Compound 19

Boc-protected amine 18 (2.90 g; 4.88 mmol) was dissolved in DCM (15 mL) and TFA (15 mL), and stirred at rt for 2 h. The mixture was concentrated, redissolved in DCM (50 mL), and washed with sat. Na₂CO₃ (40 mL). The aqueous layer was back extracted with DCM (50 mL), and the combined organic layers were dried with Na₂SO₄ and evaporated to dryness to yield a yellow foam (2.27 g; 94%). ¹H-NMR(200 MHz, CDCl₃): δ 8.07 (dd, 1H), 7.81 (dd, 1H), 7.37-7.26 (m, 11H), 6.74 (br.s, 1H), 3.74 (s, 3H), 3.59-3.46 (m, 10H), 2.81 (t, 2H), 1.68 (s, 2H) ppm. ³¹P-NMR(80 MHz, CDCl₃): δ −3.82 ppm. FT-IR (ATR): ν 3302, 2866, 1717, 1648, 1536, 1433, 1271, 1251, 1113, 744, 696 cm⁻¹.

DOTA-Building Block:

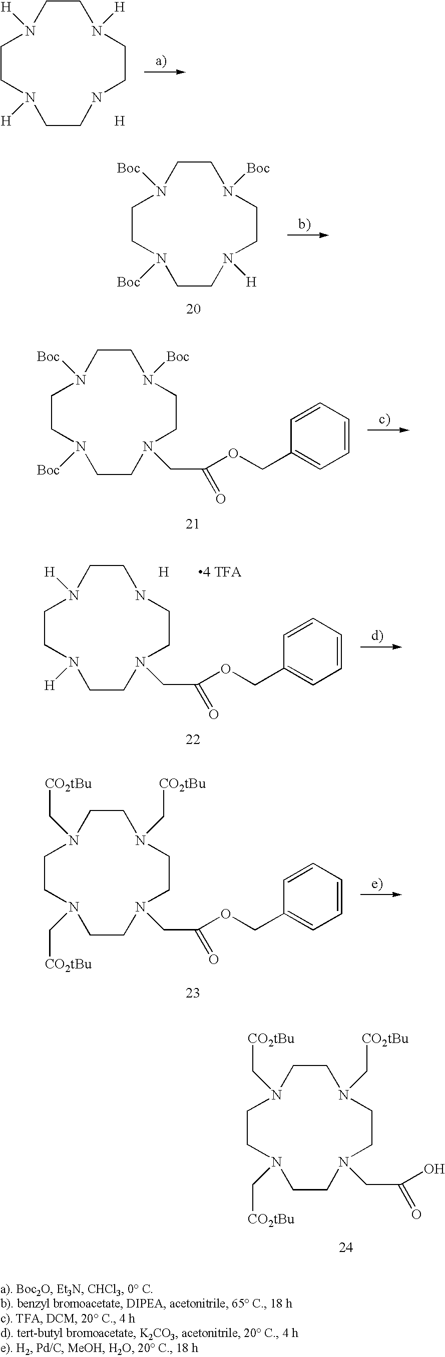

The synthesis of 20 has been reported in for example E. Kimura, J. Am. Chem. Soc., 1997, 119, 3068-3076. Compound 21 has been reported in the following Japanese patent (Japanese language). Miyake, Muneharu; Kusama, Tadashi; Masuko, Takashi. Preparation of novel nitrogen-containing cyclic compounds as NMDA receptor inhibitors, PCT Int. Appl. (2004), 57 pp. Compound 22-24 have been reported in A. Heppeler et al., Chem. Eur. J. 1999, 5, 7, 1974-1981.

Compound 20

The procedure reported in JACS, 1997, 119, 3068-3076 was followed to obtain this compound. The product was isolated by silica column chromatography using hexane-ethylacetate mixtures as eluent.

¹H NMR (CDCl₃): δ=3.6 (b, 4H, CH₂N), 3.35 (b, 1H, NH), 3.25 (b, 8H, CH₂N), 2.8 (b, 4H, CH₂N), 1.4 (s, 9H, CH₃), 1.4 (s, 18H, CH₃). ¹³C NMR (CDCl₃) δ=156-155 (multiple signals C═O), 79.8-79.1 (multiple signals C—CH₃), 51.5-48.5 and 46.5-44.5 (multiple signals CH₂—N), 28.6 (CH₃), 28.4 (CH₃). MALDI-TOF-MS: [M+H]⁺=473, [M+Na]⁺=495, [M+K]⁺=511.

Compound 21

The tri-Boc protected compound 20 (15.2 g; 32.2 mmol) was dissolved in 20 mL of acetonitrile, after which diisopropylethylamine (19 mL) and benzylbromoacetate (7.9 g; 34.5 mmol) in acetonitrile (10 mL) were added. The solution was heated to 60-65° C. and stirred overnight under an argon atmosphere. The mixture was concentrated by evaporation of the solvent, dissolved in DCM, and the solution was washed with 1 M NaOH. The organic layer was dried with Na₂SO₄, and thereafter the solvent was evaporated and co-evaporated with toluene. The pure product was isolated by silica column chromatography using hexane/ethyl acetate=1/1 as eluent. Yield: ca. 90%. ¹H NMR (CDCl₃): δ=7.4 (m, 5H, Ph-H), 5.15 (s, 2H, CH₂Ph), 3.6 (s, 2H, CH₂C(O)), 3.6-3.2 (diverse b, 12H, CH₂N), 2.9 (b, 4H, CH₂N), 1.45 (s, 9H, CH₃), 1.4 (s, 18H, CH₃) ppm. ¹³C NMR (CDCl₃): δ=170.3 (C═O ester), 156-155 (multiple signals C═O urethanes), 135.5 and 128-6-128.2 (benzene ring carbons), 79.5-79.2 (multiple signals C—CH₃), 66.2 (CH₂—O), 55.0, 53.5, 51.2, 49.9 and 47.4-46.9 (CH₂—N), 28.6 (CH₃), 28.4 (CH₃) ppm.

Compound 22

Tri-Boc protected mono benzylacetate cyclen 21 (5.4 g; 8.7 mmol) was dissolved in toluene (50 mL), and the solvent was evaporated to remove traces of water, if present. The product was then dissolved in a mixture of freshly distilled DCM (35 mL) and TFA (35 mL) and stirred under a nitrogen gas atmosphere for about 3 hours. Evaporation of the solvents (below 30° C.), redissolution in TFA (30 mL) and stirring for another 2 hours was followed by evaporation of the volatiles (below 30 ° C.). The remaining salt was stripped twice with toluene to remove TFA as much as possible to produce product 22, that was used in the next step as isolated. ¹H NMR (CDCl₃): δ=7.4 (m, 5H, Ph-H), 5.1 (s, 2H, CH₂Ph), 3.5 (s, 2H, CH₂C(O)), 3.05 (b, 4H, CH₂N), 3.0 (b, 4H, CH₂N), 2.9 (b, 4H, CH₂N), 2.8 (b, 4H, CH₂N) ppm.

Compound 23

Crude product 22, acetonitrile (50 mL) and potassium carbonate (10.6 g; 7.67 mmol) were mixed and vigorously magnetically stirred for 15 minutes, resulting in a fine suspension. Then, a solution of tert-butyl bromoacetate (6.8 g; 34.9 mmol) in acetonitrile (20 mL) was added dropwise. After stirring for 4 to 5 hours the turbid mixture was concentrated in vacuo. The residue was transferred to a separation funnel with chloroform (200 mL) and water (200 mL). The chloroform layer was separated and washed with brine. Filtration and evaporation of the chloroform gave a liquid (ca. 12 g), that was purified using silica column chromatography, by first eluting with DCM to remove contaminants such as tert-butyl bromoacetate, and then eluting with DCM/EtOH=600/30 to collect the product as a white solid (5.35 g, 93%). TLC: Rf=0.23 (DCM/EtOH=8/1), coloration with I₂ in an aqueous 10% KI-solution. ¹H NMR (CDCl₃): δ=7.4-7.3 (m, 5H, Ph-H), 5.10 (s, 2H, CH₂Ph) 3.6-2.0 (broad, 24H, cyclen ring N—CH₂ and CH₂COOR), 1.45 (s, 9H, C(CH₃)₃), 1.40 (s, 18H, C(CH₃)₃) ppm. ¹³C NMR (CDCl₃): δ=173.5, 172.9 and 172.8 (C═O), 135.0, 128.6, 128.5 and 128.3 (benzene ring), 81.9 (C(CH₃)₃), 66.8 (CH₂O), 55.8, 55.7 and 54.9 (CH₂—C(O)), 52.3 and 48.7 (both very broad, CH₂N), 27.8 (C(CH₃)₃) ppm. FT-IR (cm⁻¹): ν=2977-2830 (w), 1724 (vs, C═O esters), 1368 (vs), 1227 (vs), 1157 (vs), 1105 (vs) cm⁻¹. MALDI-TOF-MS: [M+H]⁺=663 Da and [M+Na]⁺=685 Da.

Compound 24

Benzylester 23 (5.3 g; 7.99 mmol) was dissolved in methanol (100 mL) and demineralized water (50 mL) and the solution was transferred to a thick walled Parr hydrogenation reaction vessel. Nitrogen gas was bubbled through the solution and the Pd/C 10% catalyst (305 mg) was added. The mixture was shaken at a 70 psi hydrogen gas overpressure for the duration of 4 hours. The mixture was filtered, and the filtrate concentrated in vacuo. Remaining water was removed by coevaporation with acetonitrile (40 mL). The product was further dried and stored in a vacuum desiccator over P₂O₅. to yield a foamy light-yellow solid (4.33 g; 95%). The product is very hygroscopic. ¹H NMR (CDCl₃): δ=3.7-3.2, 3.2-2.6 and 2.6-2.0 (all three very broad and overlapping, CH₂N), 1.41 (s, 18H, (CH₃)₃), 1.39 (s, 9H, C(CH₃)₃) ppm. ¹³C NMR (CDCl₃): δ=174.2 and 172.3 (C═O), 82.1 and 81.9 (C(CH₃)₃), 55.9, 55.7 and 55.3 (CH₂—C(O)), 53.5-50.5 and 50.5-47.0 (both very broad, CH₂N), 27.9 and 27.7 (C(CH₃)₃) ppm. FT-IR (cm⁻¹) ν=2976 (w, CH-stretch), 2824 (w; CH-stretch), 1725 (vs, C═O, stretch esters and acid) cm⁻¹. MALDI-TOF-MS: [M+H]⁺=573 Da and [M+Na]⁺=595 Da.

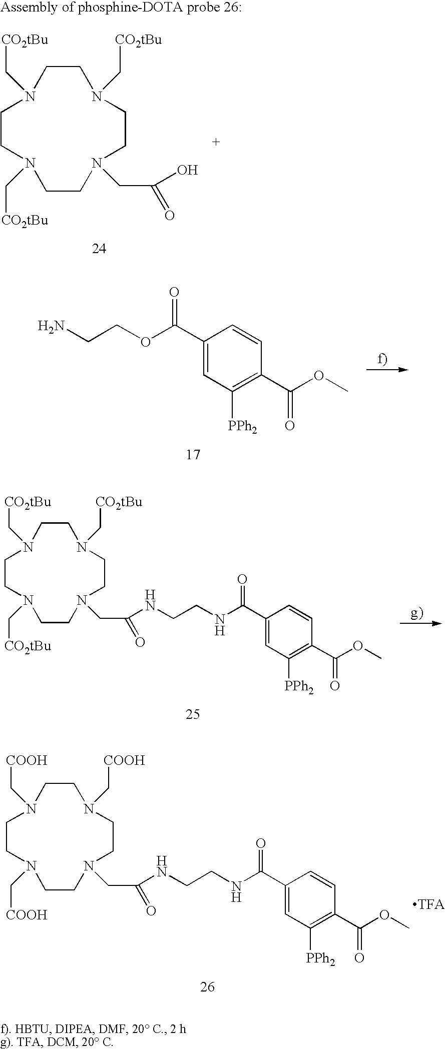

Compound 25

HBTU (0.664 g; 1.75 mmol) was dissolved in dry DMF (7 mL) and DIPEA (0.45 g; 3.50 mmol) and carboxylic acid 24 (1.00 g; 1.75 mmol) were added. The mixture was stirred under argon at rt for 10 min, and amine 17 (0.85 g; 2.10 mmol) was added. The mixture was stirred for 2 h, subsequently diluted with ether (200 mL), and washed with sat. NaHCO₃ (3 times 100 mL). The organic layer was dried with Na₂SO₄, and concentrated. The crude product was purified by column chromatography on silica, with CHCl₃ as the initial eluent, then 4% MeOH in CHCl₃. The product was obtained as a yellow foam (900 mg; 55%). ¹H-NMR(200 MHz, CDCl₃): δ 8.06 (dd, 1H), 7.86 (dd, 1H), 7.53 (dd, 1H, 7.32 (m, 10H), 7.02 (br.t, 1H), 6.62 (br.t, 1H), 3.73 (s, 3H), 3.6-2.0 (br.m, 28H), 1.46 (s, 9H), 1.40 (s, 18H) ppm. ³¹P-NMR(80 MHz, CDCl₃): δ −3.97 ppm. FT-IR (ATR): ν 3426, 2978, 2826, 1724, 1677, 1524, 1369, 1228, 1159, 1107, 837, 734 cm⁻¹. MALDI-TOF: m/z [M+H]⁺ 961.4 Da; [M+Na]⁺ 983.4 Da.

Compound 26

Tri-tBu-protected 25 was dissolved in DCM (6 mL) and TFA (6 mL), and stirred at rt for 1 h. The mixture was concentrated, and again dissolved in DCM (6 mL) and TFA (6 mL), and stirred for an additional 2 h. The mixture was concentrated and coevaporated with CHCl₃ for 2 times, and then thoroughly dried in vacuo. The product was dissolved in H₂O (30 ml) and lyophilized to yield a fluffy, yellow powder (541 mg, 94%). ¹H-NMR(200 MHz, MeOD): δ 8.05 (dd, 1H, J=3.6, 8.2), 7.84 (dd, 1H, J=1.6, 8.2), 7.51 (dd, 1H, J=3.6, 1.6), 7.37-7.23 (m, 10H), 4.1-3.7 (br.s, 8H), 3.67 (s, 3H), 3.6-3.1 (br.m, 20H) ppm. ³¹P-NMR(80 MHz, MeOD): δ 34.34 (small, phosphine oxide), −3.80 (large, product), −15.20 (t, HPO₂F₂, J=953) ppm. FT-IR (ATR): ν 3301, 3073, 2856, 2541, 1717, 1667, 1539, 1435, 1292, 1188, 1131, 720, 698 cm⁻¹. MALDI-TOF: m/z [M+H]⁺ 793.3 Da.

PS: the impurity difluorophosphoric acid (HPO₂F₂) originates from the hexafluorophosphate anion (PF₆⁻), which partly remains present in the sample after the coupling reaction with HBTU. Under acid conditions, this anion is converted to difluorophosphoric acid. (Kolditz, L. Z. Anorg. Chem. 1957, 293, 155-167).

Assembly of Phosphine-DOTA Probe 28:

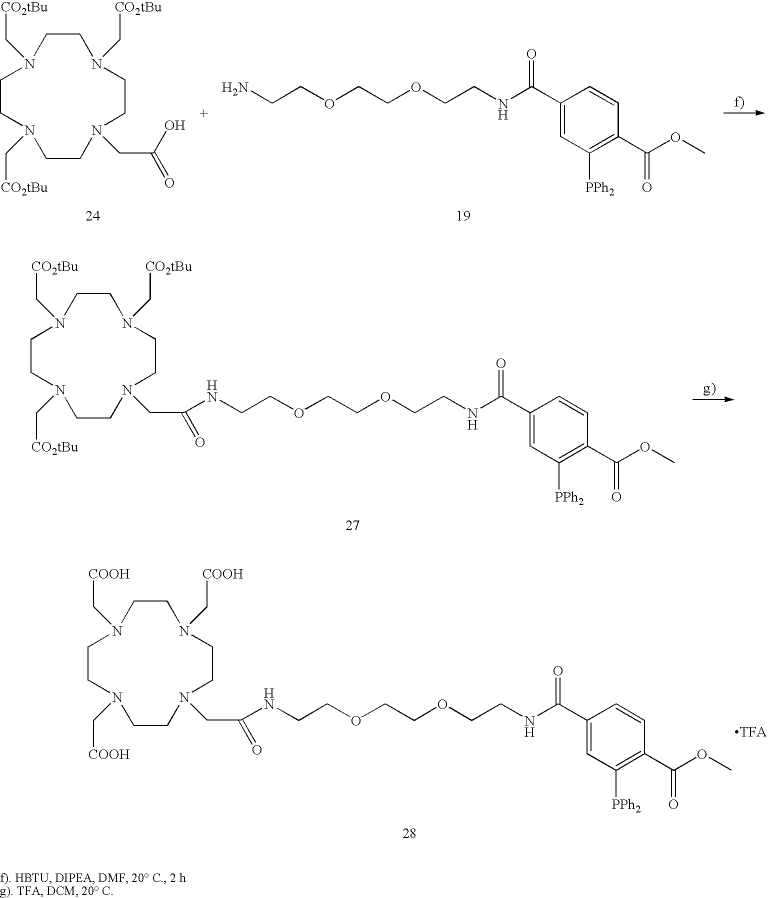

Compound 27

HBTU (1.32 g; 3.49 mmol) was dissolved in dry DMF (15 mL) and DIPEA (0.90 g; 6.98 mmol) and carboxylic acid 24 (2.00 g; 3.49 mmol) were added. The mixture was stirred under argon at rt for 10 min, and a solution of amine 19 (1.90 g; 3.84 mmol) in DMF (5 mL) was added. The mixture was stirred for 2 h, concentrated, redissolved in DCM (100 mL), and subsequently washed with sat. NaHCO₃ (3 times 50 mL), H₂O (3 times 50 mL), and 1 M NaOH (50 mL). The organic layer was dried with Na₂SO₄, and concentrated. The crude product was purified by column chromatography on silica, with CHCl₃ as the initial eluent, then 5% MeOH in CHCl₃. The product was obtained as a yellow foam (1.21 g; 33%). ¹H-NMR(200 MHz, CDCl₃): δ 8.05 (dd, 1H), 7.81 (dd, 1H), 7.41-7.27 (m, 11H), 6.52 (br.m, 2H), 3.74 (s, 3H), 3.60 (m, 10H), 3.39 (t, 2H), 3.3-2.0 (br.m, 24H), 1.45 (s, 27H) ppm. ³¹P-NMR(80 MHz, CDCl₃): δ −3.82 ppm. FT-IR (ATR): ν 3424, 2976, 2827, 1724, 1674, 1536, 1369, 1228, 1161, 1106, 838, 749 cm⁻¹. LC-MS: m/z [M+H]⁺ Calcd. 1049.2 Da, Obsd. 1049.5 Da.

Compound 28

Tri-tBu-protected 27 was dissolved in DCM (7 mL) and TFA (5 mL), and stirred at rt for 1 h. The mixture was concentrated, and again dissolved in DCM (7 mL) and TFA (5 mL), and stirred for an additional 2 h. The mixture was concentrated and coevaporated with CHCl₃ for 2 times, and then thoroughly dried in vacuo. The product was dissolved in H₂O (30 ml) and lyophilized to yield a fluffy, yellow powder (1.16 g, 96%). ¹H-NMR(200 MHz, MeOD): δ 8.04 (dd, 1H, J=3.7, 8.1), 7.79 (dd, 1H, J=1.7, 8.1), 7.45 (dd, 1H, J=3.7, 1.7), 7.4-7.2 (m, 10H), 4.05-3.7 (br.m, 8H), 3.68 (s, 3H), 3.6-3.1 (br.m, 28H) ppm. ³¹P-NMR(80 MHz, MeOD): δ 33.82 (small, phosphine oxide), −3.91 (large, product), −15.19 (t, HPO₂F₂, J=953) ppm. FT-IR (ATR): ν 3287, 3074, 2872, 2546, 1718, 1651, 1547, 1435, 1292, 1189, 1130, 745, 720, 698 cm⁻¹. MALDI-TOF: m/z [M+H]⁺ 881.4 Da.

Synthesis of Complex of Probe 28 with Eu (29):

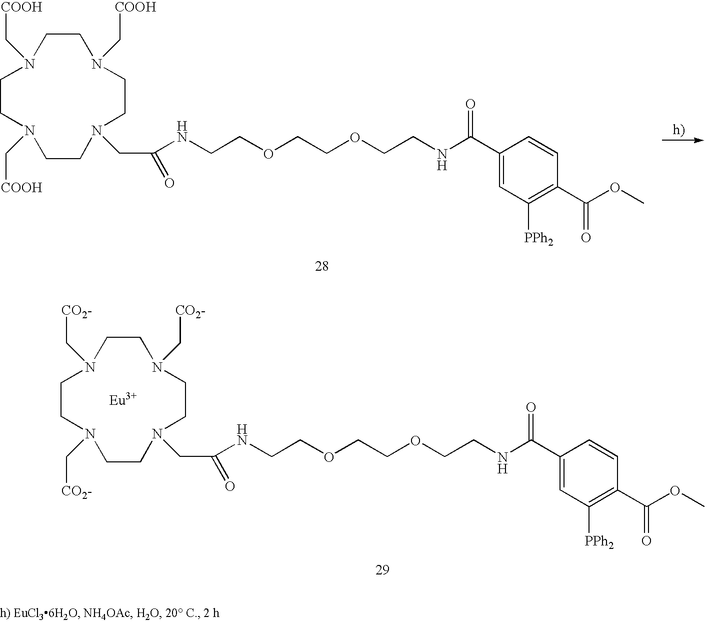

Eu³⁺-complex 29

Compound 28 (100 mg; 0.114 mmol) and ammonium acetate (87.9 mg; 1.14 mmol) were dissolved in degassed, ultrapure H₂O (60 mL). EuCl₃.6H₂O (37.4 mg; 0.102 mmol) was added, and the mixture, which was slightly turbid, was stirred under Ar at rt for 2 h. Then, it was filtered and lyophilized to yield the complex as a yellow solid (123 mg). ¹H-NMR(200 MHz, H₂O): δ 7.8-7.0 (br.m), 3.6-2.4 (br.m) ppm. ³¹P-NMR(80 MHz, D₂O): δ 37.02 (small, phosphine oxide), −5.30 (large, product), −15.01 (t, HPO₂F₂, J=962) ppm. FT-IR (ATR): ν 3050, 1664, 1590, 1403, 1292, 1128, 721 cm⁻¹. MALDI-TOF: m/z [M+H]⁺ 1031.1 Da.

### Example 12

### Water Soluble Azide With Active Ester (34) for the Synthesis of Azide-Functionalized Targeting Probes

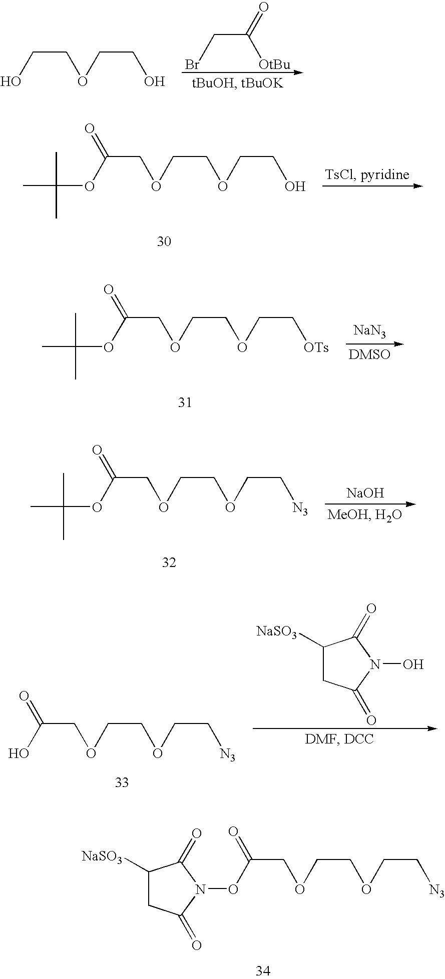

Experimental

See Example 11.

Compound 30

tert-BuOK (12.0 g; 107 mol) was added in portions to a stirred mixture of diethylene glycol (27.0 g; 0.254 mol) and tert-butanol (110 mL). The solution was kept under argon and stirred in a warm water bath (ca. 40° C.) to solubilize the potassium salt. After 90 min the solution was cooled to 15° C., and tert-butyl bromoacetate (26.8 g; 0.137 mol) was added over 5 min. A precipitate (KBr) formed, and the mixture was stirred overnight at room temperature. The solvents were evaporated, the mixture was dissolved in water, and then the aqueous layer was extracted with ether/pentane to remove contaminants. Extraction with DCM (3 times) collected the product. The organic layer was dried with Na₂SO₄ and concentrated. The crude product was purified by silica column chromatography using a gradient of hexane/dimethoxyethane mixtures. The product was obtained as a colorless oil (yield 25%). TLC (silica, hexane/dimethoxyethane 1/1): Rf=0.30. ¹H NMR (CDCl₃): δ=3.95 (s, 2H, CH₂C(O)), 3.7-3.6 (6H), 3.55 (t, 2H), 2.75 (t, 1H, OH, 1.4 (s, 9H, CH₃), ppm. ¹³C NMR (CDCl₃): δ=169.5 (C═O), 81.5 (CCH₃), 72.5, 70.7, 70.2 and 68.8 (CH₂O), 61.5 (CH₂OH), 27.9 (CH₃) ppm.

Compound 31

Tosyl chloride (11.9 g; 62.4 mmol) was added in portions to a stirred and ice-cooled mixture of alcohol 30 (11.7 g; 52.9 mmol) and dry pyridine (30 mL). The solution was stirred overnight under an argon atmosphere at 4° C. resulting in the development of a precipitate (pyridinium chloride). Ice water was added to the mixture which was subsequently stirred for 5 min to hydrolyse excess of tosyl chloride. Extraction of the aqueous layer with diethylether (3 times), washing of the combined organic layers with a cold HCl solution and with cold water, was followed by drying of the ether with Na₂SO₄. Evaporation of the solvent gave the oily product (16 g, 80%). ¹H NMR (CDCl₃): δ=7.8 (m, 2H, Ar—H) and 7.3 (m, 2H, Ar—H), 4.1 (t, 2H), 3.95 (s, 2H, CH₂C(O)), 3.7-3.5 (6H), 2.4 (s, 3H, Ar—CH₃) 1.4 (s, 9H, CH₃) ppm. ¹³C NMR (CDCl₃): δ=169.5 (C═O), 144.7, 132.9, 129.7 and 127.9 (benzene ring carbons), 81.5 (CCH₃), 70.6, 70.5, 69.1, 68.9 and 68.6 (CH₂O), 28.0 (CH₃), 21.5 (Ar—CH₃) ppm.

Compound 32

The tosylate 31 (15 g; 40.0 mmol) was added to a solution of NaN₃ (3.20 g; 49.2 mmol) in dimethylsulfoxide (90 mL). The mixture was stirred for 2 d, while keeping it under an argon atmosphere. Addition of water, extraction with ether (3 times), washing of the combined organic layers with water, drying with Na₂SO₄ and concentration gave the product as an oil (9.9 g; 100%). ¹H NMR (CDCl₃): δ=3.95 (s, 2H, CH₂C(O)), 3.7-3.6 (6H), 3.4 (t, 2H, CH₂N₃), 1.4 (s, 9H, CH₃) ppm. ¹³C NMR (CDCl₃): δ=169.5 (C═O), 81.5 (CCH₃), 70.7, 70.6, 69.9 and 69.0 (CH₂O), 50.6 (CH₂N₃), 28.0 (CH₃) ppm.

Compound 33

The azide 32 (9.9 g; 40.0 mmol) was added to a stirred solution of water (10 mL), MeOH (50 mL) and NaOH (2.05 g; 51.3 mmol), and the resulting turbid mixture was stirred overnight at room temperature. The methanol was evaporated after addition of some HCl-solution to lower the pH of about 7. Water was added and the product was extracted using several portions of DCM. The combined organic layers were washed with a small amount of water and then dried with Na₂SO₄. Concentration of the solution gave a nearly colorless oily product (7.3 g; 95%). ¹H NMR (CDCl₃): δ=10.0 (b, 1H, COOH), 4.2 (s, 2H, CH₂C(O)), 3.8-3.6 (6H), 3.4 (t, 2H, CH₂N₃) ppm. ¹³C NMR (CDCl₃): δ=174.5 (C═O), 71.1, 70.4, 70.0 and 68.3 (CH₂O), 50.5 (CH₂N₃) ppm.

Compound 34

The acid 33 (310 mg; 1.64 mmol), sodium N-hydroxy sulfo-succinimide (316 mg; 1.46 mmol), DCC (640 mg; 3.11 mmol) and 3 mL of dry DMF were mixed and the resulting suspension was sonicated in a water bath for an hour at 50° C. and then stirred for three days at room temperature. Before filtering over a glass filter and washing with a little dry DMF and acetonitrile, the mixture was kept at 4° C. for several hours. Dry ethyl acetate (30 mL) and ether (200 mL) were added to the clear filtrate, producing a milky suspension. Centrifugation gave a white powder to which ether was added; again the suspension was centrifuged. The powder was dried in vacuo. Yield: 200 mg (35%).

Caution: Keep the compound dry as it is somewhat hygroscopic and hydrolyses back to the starting compounds!

1. A kit for targeted medical imaging or therapeutics comprising:
at least one targeting probe comprising a primary targeting moiety and a secondary target; and at least one further probe selected from either:
an imaging probe comprising a secondary targeting moiety and a label; or
a therapeutic probe comprising a secondary targeting moiety and a pharmaceutically active compound,
characterized in that one of the targeting probe or the imaging or therapeutic probe comprises, as secondary target and secondary targeting moiety respectively, either at least one azide group and in that the other probe comprises at least one phosphine group, said phosphine and said azide groups being reaction partners for the Staudinger ligation.

2. The kit according to claim 1 wherein the targeting probe comprises the at least one azide group and wherein the imaging or therapeutic probe comprises the at least one phosphine group.

3. The kit according to claim 1, wherein the primary targeting moiety binds to a component within the vascular system.

4. The kit according to claim 1, wherein the primary targeting moiety binds to a receptor.

5. The kit according to claim 1, wherein the primary targeting moiety binds to an intracellular component.

6. The kit according to claim 1, wherein the primary targeting moiety is an antibody.

7. The kit according to claim 1, wherein the primary targeting moiety is cell-permeable.

8. The kit according to claim 1, which comprises an imaging probe.

9. The kit according to claim 8, wherein said imaging probe comprises a detectable label which is a contrast agent used in traditional imaging systems, selected from the group consisting of MRI-imageable agents, spin labels, optical labels, ultrasound-responsive agents, X-ray-responsive agents, radionuclides, and FRET-type dyes.

10. The kit according to claim 8, wherein said detectable label is selected from the group consisting of (bio)luminescent or fluorescent molecules or tags, radioactive labels, biotin, paramagnetic or supermagnetic imaging reagents.

11. The kit according to claim 8, wherein said label comprises a radionuclide selected from the group consisting of ³H, ¹¹C, ¹³N, ¹⁵O, ¹⁸F, ⁵¹Cr, ⁵²Fe, ^{52m}Mn, ⁵⁵Co, ⁶⁰Cu, ⁶¹Cu, ⁶²Zn, ⁶²Cu, ⁶³Zn, ⁶⁴Cu, ⁶⁶Ga, ⁶⁷Ga, ⁶⁸Ga, ⁷⁰As, ⁷¹As, ⁷²As, ⁷⁴As, ⁷⁵Se, ⁷⁵Br, ⁷⁶Br, ⁷⁷Br, ^{80m}Br, ^{82m}Br, ⁸²Rb, ⁸⁶Y, ⁸⁸Y, ⁸⁹Sr, ⁸⁹Zr, ⁹⁷Ru, ^{99m}Tc, ¹¹⁰In, ¹¹¹In, ^{113m}In, ^{114m}In, ^{117m}Sn, ¹²⁰I, ¹²²Xe, ¹²³I, ¹²⁴I, ¹²⁵I, ¹⁶⁶Ho ¹⁶⁷Tm, ¹⁶⁹Yb ^{193m}Pt, ^{195m}Pt, ²⁰¹Tl, ²⁰³Pb.

12. The kit according to claim 8, wherein said label comprises a paramagnetic ion selected from the group consisting of Gd, Fe, Mn, Cr, Co, Ni, Cu, Pr, Nd, Yb, Tb, Dy, Ho, Er, Sm, Eu, Ti, Pa, La, Sc, V, Mo, Ru, Ce, Dy, Tl.

13. The kit according to claim 8, wherein said label is a small size organic PET (Positron Emission Tomography) or SPECT label.

14. The kit according to claim 8, wherein the imaging probe further comprises a pharmaceutically active compound.

15. The kit according to claim 1, which comprises a therapeutic probe.

16. An imaging probe comprising a secondary targeting moiety and a label characterized in that said imaging probe comprises as secondary target at least one azide group or at least one phosphine group, said phosphine or said azide groups being suitable reaction partners for the Staudinger ligation and in that said label is an imaging label.

17. The imaging probe according to claim 16, wherein said label is selected from the group consisting of a metal particle, an organic PET or SPECT labeled prosthetic group, an optical label, a metal complex for PET, SPECT or MRI, a microbubble, or an Iodine- or Barium-comprising molecule or vesicle.

18. Pharmaceutical composition comprising the imaging probe according to claim 16.

19. Method for the preparation of a diagnostic composition for imaging, comprising using the imaging probe according to claim 16.

20. The use of a targeting probe comprising a primary targeting moiety and a secondary target, characterized in that said targeting probe comprises as said secondary target at least one azide group or at least one phosphine group, said phosphine or said azide groups being suitable reaction partners for the Staudinger ligation, as a tool in targeted medical imaging.

21. The use of a targeting probe comprising a primary targeting moiety and a secondary target, characterized in that said targeting probe comprises as said secondary target at least one azide group or at least one phosphine group, said phosphine or said azide groups being suitable reaction partners for the Staudinger ligation, in the manufacture of a tool for medical imaging.

22. An imaging probe comprising an imaging agent for MRI and a phosphine group, which can react with an azide in a Staudinger ligation, characterized in that a metal atom of the imaging agent is co-ordinated with carboxylic acid or acids via a link containing the phosphine group.

23. An imaging probe comprising a PET or SPECT label and triphenylphosphine as a secondary targeting moiety.

24. An imaging probe according to claim 22 with general structure (6) 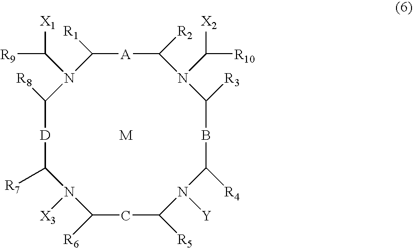
wherein M is a paramagnetic metal ion selected from the group consisting of Gd(III), Fe(III), Mn(II), Yt(III), Cr(III), Eu(III), Yb (III) and Dy(III); A, B, C and D are either single bonds or double bonds; X₁, X₂, and X₃ are —OH, —COO—, —CH₂OH—, CH₂COO—; R₁-R₁₀ are hydrogen, alkyl, aryl, phosphorus moiety,
and wherein Y is a substituted triphenyl phosphine such as (CH₂)₂—COO—C₆H₄N(CH₂COOH)₂—P—(C₆H₅)₂ or another substituted triphenyl phosphine which allows one or more coordinations between the substituted phosphine and the paramagnetic metal.

25. An imaging probe according to claim 24 wherein the paramagnetic metal is Gd.

26. A compound according to claim 24 represented by formula (1)

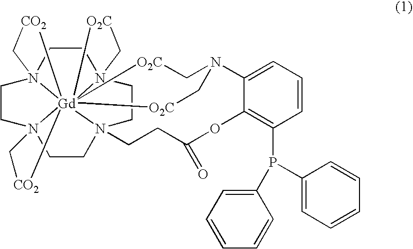

27. A combined probe for medical imaging comprising a primary targeting moiety and a detectable label characterized in that the said targeting moiety is connected to the detectable label via an amide bond or a triphenylphosphine oxide moiety.

28. The combined probe according to claim 27, wherein the detectable label is an imaging agent selected from the group consisting of a metal particle, an organic PET or SPECT labelled prosthetic group, an optical label, a metal complex for PET, SPECT or MRI, a microbubble and an Iodine- or Barium-comprising molecule or vesicle.

29. A method of in vitro preparing a combined targeting and imaging or therapeutic probe, comprising a primary targeting moiety and a detectable label or a pharmaceutically active agent, comprising the step of reacting a phosphine comprising detectable label with an azide-comprising primary targeting moiety or reacting an azide-comprising detectable label with an phosphine-comprising primary targeting moiety.

30. The method according to claim 29, wherein the imaging agent is selected from the group of an organic PET or SPECT labelled prosthetic group, an optical label, a metal complex for PET, SPECT or MRI, a microbubble and an Iodine- or Barium-comprising molecule or vesicle.

31. A method of developing a targeting probe with optimal binding affinity for a target and optimal reaction with an imaging or therapeutic probe, which comprises
a) making a compound library of the targeting moiety of said targeting probe, whereby the secondary target is introduced at different sites on said targeting moiety
b) screening the so obtained compound library for binding with the target and with an imaging and/or targeting probe.

32. A library of derivatives of a specific peptide characterized in that said derivatives are modified with an azide group at different amino acid positions in the peptide chain of said peptide.

33. Use of a library according to claim 32 for determining the binding properties to a primary target of an azide-containing peptide and/or of the reaction product of an azide comprising peptide and a phosphine comprising imaging or therapeutic probe.

34. A kit for targeted medical imaging or therapeutics comprising:
at least one target metabolic precursor comprising a secondary target; and at least one further probe selected from either
an imaging probe comprising a secondary targeting moiety and a label; or
a therapeutic probe comprising a secondary targeting moiety and a pharmaceutically active compound
characterized in that one of the target metabolic substrate or the imaging or therapeutic probe comprises, as secondary target and secondary targeting moiety respectively, either at least one azide group and in that the other probe comprises at least one phosphine group, said phosphine and said azide groups being reaction partners for the Staudinger ligation.

35. The kit according to claim 34 wherein the target metabolic precursor comprises the at least one azide group and wherein the imaging or therapeutic probe comprises the at least one phosphine group.

36. The kit according to claim 34, wherein said metabolic precursor is selected from a group consisting of sugars, amino acids, nucleobases, and choline.

37. A kit for targeted medical imaging or therapeutics comprising:
at least one reporter probe comprising a secondary target; and at least one further probe selected from either
an imaging probe comprising a secondary targeting moiety and a label; or
a therapeutic probe comprising a secondary targeting moiety and a pharmaceutically active compound
characterized in that one of the reporter or the imaging or therapeutic probe comprises, as secondary target and secondary targeting moiety respectively, either at least one azide group and in that the other probe comprises at least one phosphine group, said phosphine and said azide groups being reaction partners for the Staudinger ligation.

38. The kit according to 34, which comprises an imaging probe.

39. The kit according to claim 38, wherein said imaging probe comprises a detectable label which is a contrast agent used in traditional imaging systems, selected from the group consisting of MRI-imageable agents, spin labels, optical labels, ultrasound-responsive agents, X-ray-responsive agents, radionuclides, and FRET-type dyes.
